(19) United States
(12) Patent Application Publication
(10) Pub. No.: US 2026/0191399 A1
(43) Pub. Date: Jul. 9, 2026

(54) ENDOSCOPE

(71) Applicant: OLYMPUS MEDICAL SYSTEMS CORP., Tokyo (JP)

(72) Inventors: Akihiro KATO, Tokyo (JP); Taizo SHINGA, Tokyo (JP)

(73) Assignee: OLYMPUS MEDICAL SYSTEMS CORP., Tokyo (JP)

(21) Appl. No.: 19/557,475

(22) Filed: Mar. 5, 2026

Related U.S. Application Data
(63) Continuation of application No. PCT/JP2024/027413, filed on Jul. 31, 2024.
(60) Provisional application No. 63/536,782, filed on Sep. 6, 2023.

(51) Int. Cl. A61B 1/00

(52) U.S. Cl. CPC A61B 1/00087

(57) ABSTRACT

The endoscope includes a cylinder with an inner space, a first moving member with a first connecting member having at least two protrusions, a second moving member with a second connecting member including a head and a narrower neck, and an opening/closing mechanism. The opening/closing mechanism changes the first connecting member between a closed state to hold the head portion and an open state to release it, and includes a proximal first part with a smaller cross-sectional area that restricts the first connecting member from releasing the second connecting member, and a distal second part with a larger cross-sectional area that allows the first connecting member to open. By moving along the axis within these parts, the members transition between holding and releasing states. The first and second parts form at least a part of the inner space. The opening/closing mechanism forms at least a part of the cylinder.

### CROSS REFERENCE TO RELATED APPLICATIONS

This application is a continuation of International Application No. PCT/JP2024/027413, filed on July 31, 2024, which claims the benefit of U.S. Provisional Patent Application No. 63/536,782, filed on September 6, 2023. The entire disclosures of the above-referenced applications are incorporated herein by reference.

### FIELD

The present disclosure relates to an endoscope which includes, at a distal end portion of an insertion portion, a treatment instrument raising base, the endoscope further including a treatment instrument raising wire connection mechanism for enabling attachment and detachment of a treatment instrument raising wire, the treatment instrument raising wire being configured to contribute to a raising and lowering operation of the treatment instrument raising base.

### BACKGROUND

Conventionally, endoscopes have been widely used in medical fields and industrial fields, for example. Medical endoscopes used in the medical fields are formed by including an insertion portion and an operation portion. The insertion portion is formed in an elongated tubular shape and includes, at a distal end portion thereof, an image pickup unit, etc. The operation portion is provided continuously with a proximal end side of the insertion portion and includes, on an outer surface thereof, various operation members, etc.

Conventional endoscopes of this type have functions for acquiring an image inside an organ or the like by inserting the insertion portion into a living body (subject) and for displaying and recording the acquired image. A user performs observation and examination of a lesion part and the like in the organ based on the image acquired and displayed by the endoscope.

In addition, some of the conventional endoscopes of this type have a configuration in which the insertion portion includes a conduit (a so-called treatment instrument insertion conduit) through which a treatment instrument or the like is inserted, and the treatment instrument is used by causing a distal end part of the treatment instrument inserted through the conduit to protrude outward from a distal end of the conduit. Furthermore, some of the endoscopes having such a configuration include, at the distal end portion of the insertion portion thereof, a treatment instrument raising base for changing a protruding direction of the treatment instrument.

The treatment instrument is an instrument which includes, at a distal end thereof, a forceps or the like and which is used for performing various kinds of treatment such as biopsy for collecting a tissue sample in a body, incision of a lesion part in the body, etc.

In addition, the treatment instrument raising base is driven in response to an operation of operation members (an operation lever and the like) provided at the operation portion, and configured to be capable of performing a raising operation or falling operation of the treatment instrument. The treatment instrument raising base is provided at the distal end portion of the insertion portion of the endoscope. A distal end of the treatment instrument raising wire is connected to the treatment instrument raising base. A proximal end side of the treatment instrument raising wire is connected to the operation lever provided at the operation portion. The treatment instrument raising wire is a member formed of a flexible elongated linear member, arranged so as to be inserted through the inside of the insertion portion, and configured to be slidable in a longitudinal axis direction of the insertion portion.

Such a configuration achieves a configuration of the treatment instrument raising base for performing a predetermined raising and lowering operation in response to a predetermined operation performed on the operation lever.

For the conventional endoscope including such a treatment instrument raising base, Japanese Patent Application Laid-Open Publication No. 2022-38181, for example, proposes various mechanisms (or arrangements) for fixing an end portion of the treatment instrument raising wire.

In addition, in recent years, various types of endoscopes provided with a treatment instrument raising base have been proposed, and for example, European Patent No. EP1759626 B1, etc., proposes the endoscope in which a treatment instrument raising wire is configured to be attachable and detachable. To achieve such a configuration, the conventional endoscopes of such a type are provided with various mechanisms for fixing or releasing an end portion of the treatment instrument raising wire to a predetermined member in an attachable/detachable manner.

### SUMMARY

According to aspects of the present disclosure, an endoscope is provided, which includes a cylinder, a first moving member, a second moving member, and an opening/closing mechanism. The cylinder includes an inner space formed along an axis extending from a distal end toward a proximal end. The first moving member is configured to move in the inner space along the axis and including a first connecting member configured to be openable and closable. The first connecting member includes at least two protrusions. The second moving member is configured to move in the inner space along the axis and includes a second connecting member configured to be held by the first connecting member. The second connecting member includes a head portion and a neck portion. The head portion is configured to be held by the at least two protrusions. The neck portion is disposed closer to the distal end than the head portion is and formed to be narrower than the head portion. The opening/closing mechanism is configured to change a state of the first connecting member between an open state where the first connecting member is opened to release the second connecting member and a closed state where the first connecting member is closed to hold the second connecting member. The opening/closing mechanism includes a first part and a second part. The first part has a first cross-sectional area adapted in size to restrict the first connecting member from releasing the second connecting member. The second part is disposed closer to the distal end than the first part is. The second part has a second cross-sectional area that is larger than the first cross-sectional area and adapted in size to allow the first connecting member to release the second connecting member. The first part and the second part form at least a part of the inner space. The opening/closing mechanism forms at least a part of the cylinder.

### BRIEF DESCRIPTION OF DRAWINGS

FIG. 1 is an exterior view showing a schematic configuration of an endoscope in a first embodiment of the present disclosure.

FIG. 2 is a cross-sectional view of an operation portion of the endoscope, which is cut along a virtual surface shown by an arrow reference sign [2] in FIG. 1.

FIG. 3 is an exterior perspective view showing by taking out only a treatment instrument operation lever in the endoscope in FIG. 1.

FIG. 4 is a main-part exploded perspective view showing by taking out the treatment instrument operation lever in FIG. 3 and some constituent members (a frame shaft, a link main body) in a driving mechanism of the treatment instrument operation lever.

FIG. 5 shows a working of the treatment instrument operation lever in the endoscope in FIG. 1, which shows a first state of the treatment instrument operation lever.

FIG. 6 shows the working of the treatment instrument operation lever in the endoscope in FIG. 1, which shows a second state of the treatment instrument operation lever.

FIG. 7 is a main-part enlarged cross-sectional view showing, in an enlarged manner, a region indicated by a reference sign [7] in FIG. 2.

FIG. 8 is a cross-sectional view of respective constituent members in a state where a treatment instrument raising wire connection mechanism shown in FIG. 7 is disassembled.

FIG. 9 is a cross-sectional perspective view of the treatment instrument raising wire connection mechanism shown in FIG. 7, which is partly enlarged and viewed in an oblique direction.

FIG. 10 is a main-part enlarged perspective view showing a first connecting member and a second connecting member in the treatment instrument raising wire connection mechanism shown in FIG. 7 (a state where the first connecting member is closed).

FIG. 11 is a main-part enlarged perspective view showing the first connecting member and the second connecting member in the treatment instrument raising wire connection mechanism shown in FIG. 7 (a state where the first connecting member is open).

FIG. 12 is a view showing a working of the treatment instrument raising wire connection mechanism in the endoscope in the first embodiment of the present disclosure.

FIG. 13 is a main-part enlarged cross-sectional view of a treatment instrument raising wire connection mechanism in an endoscope in a second embodiment of the present disclosure.

FIG. 14 is a main-part enlarged cross-sectional view of a treatment instrument raising wire connection mechanism in an endoscope in a third embodiment of the present disclosure.

FIG. 15 is a main-part enlarged perspective view showing a first connecting member and a second connecting member in a treatment instrument raising wire connection mechanism in an endoscope in a fourth embodiment of the present disclosure.

FIG. 16 is a main-part enlarged cross-sectional view of a treatment instrument raising wire connection mechanism in an endoscope in a fifth embodiment of the present disclosure.

### DESCRIPTION OF EMBODIMENTS

Hereinafter, the present disclosure will be described by illustrated embodiments. The respective drawings used in the following description are schematically shown, and in order to show each component in a size that enables the component to be recognized in the drawings, respective members may be shown so that the dimensional relationship, scales, etc., thereof are made different among the respective components. Therefore, the present disclosure is not limited only to the illustrated forms with respect to the number of the respective components, the shapes of the respective components, the ratio of the sizes of the respective components, the relative positional relationship of the respective components, etc., described in the respective drawings.

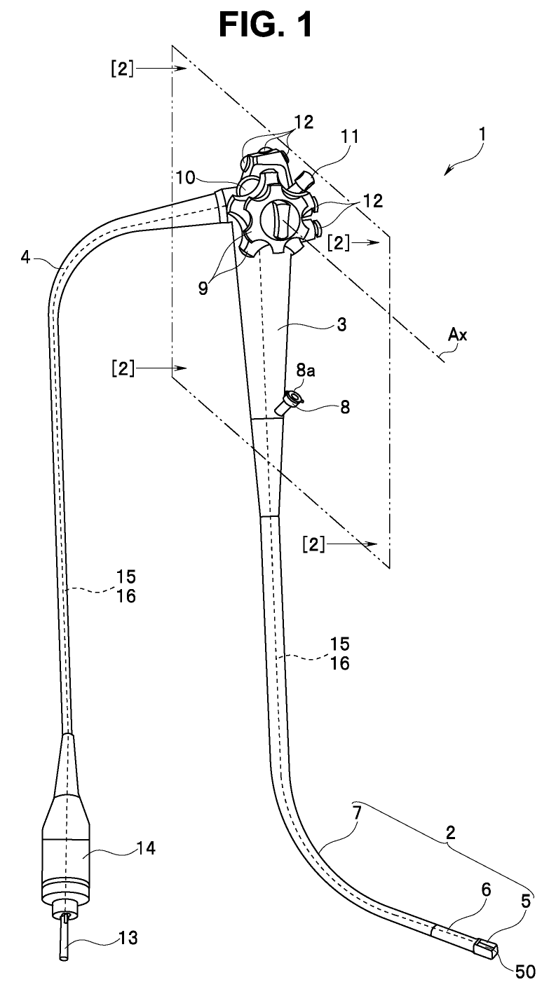

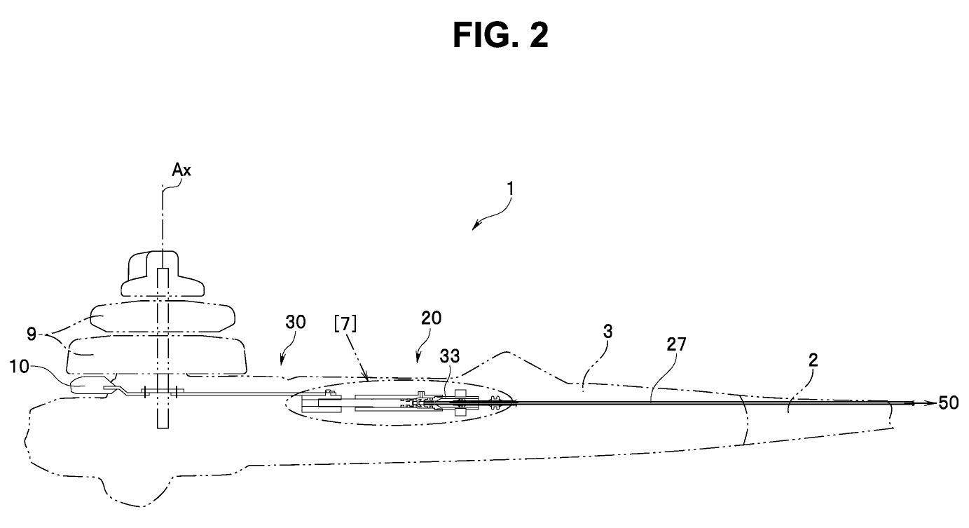

First, description will be made below on a schematic configuration of an endoscope in a first embodiment of the present disclosure with reference to FIG. 1 and FIG. 2. FIG. 1 is an exterior view showing the schematic configuration of the endoscope in the first embodiment of the present disclosure. FIG. 2 is a schematic view showing a treatment instrument raising wire connection mechanism provided in an operation portion of the endoscope in FIG. 1. Note that FIG. 2 conceptually shows a cross section of the operation portion of the endoscope cut along a virtual plane shown by an arrow reference sign [2] in FIG. 1.

As shown in FIG. 1, an endoscope 1 includes an insertion portion 2, an operation portion 3, a universal cable 4, and the like.

The insertion portion 2 is a long member formed in an elongated tubular shape and configured to be insertable into a lumen (inside of an organ, etc.) of a subject such as an organism. The insertion portion 2 includes a distal end portion 5, a bending portion 6, and a flexible tube portion 7.

Note that, in the description below, a side where the distal end portion 5 of the insertion portion 2 is located is referred to as a distal end side in the endoscope 1. In addition, a side where the operation portion 3 is located is referred to as a proximal end side in the endoscope 1.

The distal end portion 5 is disposed at the distal end of the insertion portion 2. Note that, although illustration is omitted, the distal end portion 5 is provided with an image pickup unit including an image pickup optical system, an image sensor, etc., an illumination unit including an observation optical system, etc., a gas/liquid feeding nozzle, and a gas/liquid feeding conduit, etc. In addition to the above, the distal end portion 5 is provided with a treatment instrument raising base (forceps elevator) 50.

The treatment instrument raising base 50 is a constituent unit for changing a protruding direction of a treatment instrument (not shown) which is inserted into a treatment instrument insertion conduit (not shown) and thereafter protrudes to the outside of the distal end portion 5, the treatment instrument insertion conduit being inserted and arranged in the insertion portion 2.

As shown in FIG. 2, the treatment instrument raising base 50 is connected to a treatment instrument operation lever 10 through various constituent units such as a treatment instrument raising wire 27, a treatment instrument raising wire connection mechanism 20, a driving mechanism 30 (detailed later). With such a configuration, the treatment instrument raising base 50 is rotationally moved in a predetermined direction in response to an operation input through the treatment instrument operation lever 10.

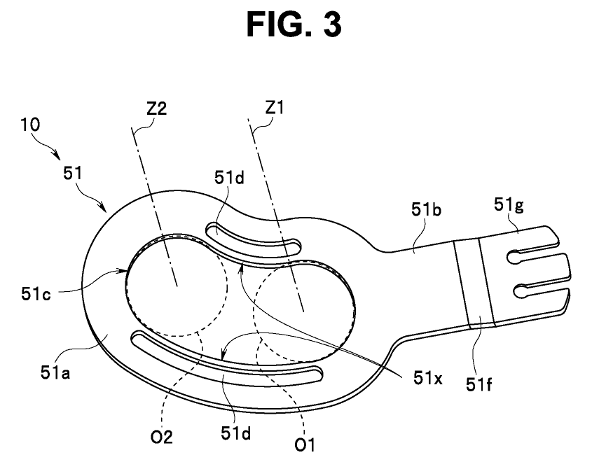

As shown in FIG. 2, the treatment instrument raising wire connection mechanism 20 and the driving mechanism 30 are disposed in the operation portion 3. In addition, the treatment instrument raising wire 27 is inserted through a treatment instrument raising wire insertion conduit 33 (not shown in FIG. 1; see FIG. 3, etc.) which is arranged so as to be inserted through the insertion portion 2 and the operation portion 3.

The bending portion 6 is provided such that the distal end thereof is continuous with the proximal end of the distal end portion 5. The bending portion 6 is a tubular member configured to be actively bendable in response to an operation input through a bending operation knob 9 to be described later.

The flexible tube portion 7 is provided such that the distal end thereof is continuous with the proximal end of the bending portion 6, and the proximal end thereof is continuous with the distal end of the operation portion 3. The flexible tube portion 7 is formed of a soft tube having flexibility.

The operation portion 3 is provided continuously with the proximal end of the insertion portion 2. In other words, the insertion portion 2 is provided on the distal end side of the operation portion 3. The operation portion 3 includes a treatment instrument insertion port 8, the bending operation knob 9, the treatment instrument operation lever 10, a bending fixing lever 11, a plurality of operation members 12, etc.

The treatment instrument insertion port 8 is an opening portion connected to the treatment instrument insertion conduit (not shown) inserted and arranged in the insertion portion 2. The treatment instrument insertion port 8 is provided with a forceps plug 8a.

The bending operation knob 9 is an operation member for performing bending operation on the bending portion 6. The bending operation knob 9 is constituted of two operation members, i.e., an operation member for up/down bending operation and an operation member for left/right bending operation. The two operation members of the bending operation knob 9 are disposed in a superimposed manner on the outer surface of the operation portion 3. In this case, the two operation members of the bending operation knob 9 are disposed rotatably around a rotation axis Ax which is the same one center axis of the two operation members. Note that the bending operation knob is also referred to as an angle knob.

The treatment instrument operation lever 10 is an operation member with which an operation force is inputted. The operation force is for performing a raising operation and a falling operation of the treatment instrument raising base 50 provided at the distal end portion 5. The treatment instrument operation lever 10 is configured to be rotationally movable within a predetermined range with the rotation axis Ax, which is the same as the rotation axis of the bending operation knob 9, as a rotation center.

The treatment instrument operation lever 10 is connected to the treatment instrument raising base 50, through the driving mechanism 30 (not shown in FIG. 1; see FIGS. 2, 3, etc., to be described later) that is disposed in the operation portion 3 and the treatment instrument raising wire 27 (not shown in FIG. 1; see FIGS. 2, 3, etc., to be described later) that is inserted through the insertion portion 2.

Such a configuration allows the operation input through the treatment instrument operation lever 10 to be transmitted to the treatment instrument raising base 50. Thus, the treatment instrument raising base 50 has a configuration capable of changing the protruding direction of the distal end of the treatment instrument (not shown) inserted and arranged in the treatment instrument insertion conduit (not shown).

Note that, as described above, the treatment instrument operation lever 10 is configured to be rotationally movable within the predetermined range with the rotation axis Ax, which is the same as the rotation axis of the bending operation knob 9, as the rotation center. In this case, the treatment instrument operation lever 10 has a mechanism for arbitrarily switching a plurality of rotational movement range settings such as a normal operation range setting adaptive to a normal use of the endoscope and a maintenance operation range setting that allows an operation in the maintenance operation range exceeding the normal operation range.

In the present embodiment, the normal operation range setting of the treatment instrument operation lever 10 allows the rotational movement within a predetermined normal operation range of the treatment instrument operation lever 10 and restricts the rotational movement exceeding the normal operation range. In addition, the maintenance operation range setting of the treatment instrument operation lever 10 allows the rotational movement exceeding the predetermined normal operation range of the treatment instrument operation lever 10. The maintenance operation range setting is used when performing a predetermined work such as a replacement work of the treatment instrument raising wire 27. A switching mechanism of the operation range settings of the treatment instrument operation lever 10 will be described later.

The bending fixing lever 11 is an operation member that acts on the bending operation knob 9, to fix the bending state of the bending portion 6 at a desired position. The bending fixing lever 11 is configured to be rotationally movable within a predetermined range with the rotation axis Ax, which is the same as that of the bending operation knob 9, as a rotation center.

Note that a bending fixing mechanism of the bending portion 6 by the bending fixing lever 11 is not directly related to the present disclosure. Therefore, detailed configuration and illustration of the configurations of the bending fixing lever 11 and the bending fixing mechanism will be omitted, supposing that they have the same configurations as those applied to the conventional endoscopes.

The plurality of operation members 12 include, for example, operation switches for performing, as appropriate, predetermined operations on image data acquired by the endoscope 1. The predetermined operations on the image data include, for example, a switching display operation between a moving image and a still image, an image enlarged display operation, an image recording operation, an image editing, and the like.

Furthermore, the plurality of operation members 12 include, for example, operation members for performing a gas/liquid feeding operation, a suction operation, etc. Here, the operation member for performing the gas/liquid feeding operation controls a feeding operation of a fluid from a fluid feeding portion (not shown) provided at the distal end portion 5. The operation member for performing the suction operation controls the pressure inside the treatment instrument insertion conduit (not shown) to a negative pressure. Such a suction operation is performed, thereby enabling, for example, mucus, etc., adhered to the distal end portion 5 to be sucked from the opening portion (not shown) of the treatment instrument insertion conduit provided at the distal end portion 5 and removed.

The universal cable 4 is a composite cable extended from one side surface of the operation portion 3. The universal cable 4 includes, at the distal end thereof, a scope connector 14. The scope connector 14 is a connector member connected to a video processor (not shown) which is an external apparatus including a light source apparatus. The scope connector 14 includes a light guide connector 13, etc., in addition to an electric contact and the like.

A light guide bundle 15, various signal lines 16, and the like are inserted through the part from the scope connector 14, the universal cable 4, the operation portion 3, to the distal end portion 5 of the insertion portion 2. The light guide bundle 15 is a constituent member that transmits a light flux emitted from the light source apparatus included in the video processor, not shown, to the distal end portion 5 of the insertion portion 2. The light flux transmitted to the distal end portion 5 by the light guide bundle 15 is emitted, as illumination light with which an observation target is irradiated, from the illumination unit (not shown) provided in the distal end portion 5.

In addition, the various signal lines 16 include, for example, an image pickup cable that transmits an image signal (image data) acquired by the image pickup unit (not shown) provided in the distal end portion 5 to the video processor (not shown), and a control signal cable that transmits a control signal outputted from the video processor to the constituent unit such as the image pickup unit. The schematic configuration of the endoscope 1 is as described above.

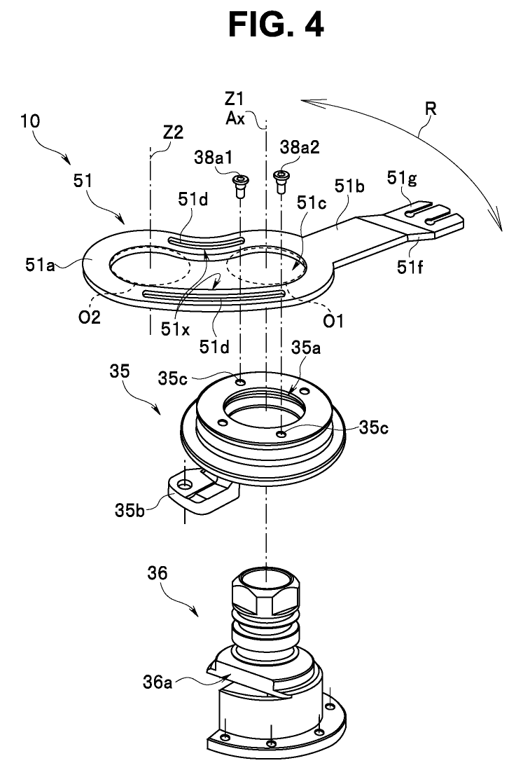

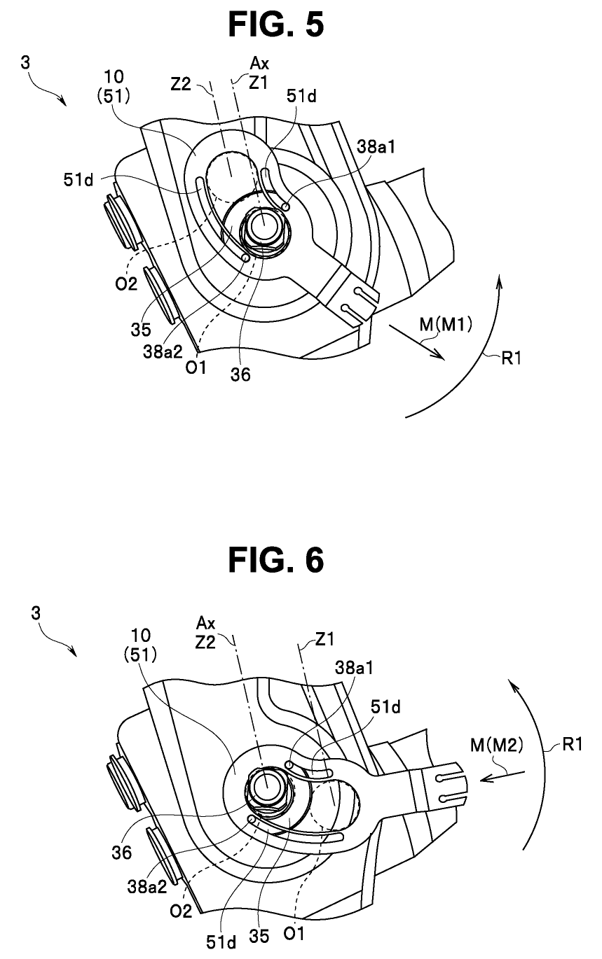

Now, description will be made below on a configuration of the switching mechanism of the operation range settings of the treatment instrument operation lever 10, with reference to FIG. 3 to FIG. 6. FIG. 3 is an exterior perspective view showing by taking out only the treatment instrument operation lever in the endoscope in FIG. 1. FIG. 4 is a main-part exploded perspective view showing by taking out the treatment instrument operation lever in FIG. 3 and some constituent members (a frame shaft, a link main body) in a driving mechanism of the treatment instrument operation lever. FIG. 5 and FIG. 6 each show a working of the treatment instrument operation lever in the endoscope of the present embodiment. Of these figures, FIG. 5 shows a first state of the treatment instrument operation lever. FIG. 6 shows a second state of the treatment instrument operation lever.

The treatment instrument operation lever 10 in the endoscope 1 of the present embodiment is an operation member that receives an operation input by a user and transmits the received operation input to the driving mechanism 30 provided in the operation portion 3. The treatment instrument operation lever 10 is rotationally movable around the rotation axis Ax which is a center axis of an inner fixing member (not shown) of the operation portion 3. The treatment instrument operation lever 10 is composed of a lever member 51 and a finger contact member (not shown).

Note that, in FIGS. 3 to 6, illustration of the finger contact member is omitted in order to avoid complicating the drawings. The configuration of the finger contact member of the treatment instrument operation lever 10 in the present embodiment is substantially the same as that of the same member in the conventional endoscopes.

The lever member 51 is formed by performing bending processing on a thin plate member made of metal, for example. As shown in FIG. 3, the lever member 51 includes a main body portion 51a, an operation arm portion 51b, a shaft insertion hole 51c, and two screw insertion slots 51d.

The main body portion 51a is formed by including the shaft insertion hole 51c, and is a part that is fixed to a link main body 35 (see FIG. 4) to be described later.

The operation arm portion 51b is a part protruding from a part of an outer peripheral edge portion of the main body portion 51a in a radially outward direction and formed in an arm shape. In the vicinity of the distal end of the operation arm portion 51b, a stepped portion 51f is provided by bending processing. The stepped portion 51f includes, at the distal end side thereof, a cover attaching portion 51g having a surface parallel to a plane of the main body portion 51a and a plane of the operation arm portion 51b. Note that the stepped portion 51f is not necessarily an essential component.

The shaft insertion hole 51c is a hole formed in a substantially center region of the main body portion 51a. The shaft insertion hole 51c is formed by connecting a first hole O1 and a second hole O2 so as to form substantially arc shapes. The shaft insertion hole 51c is a part into which a frame shaft 36 is inserted and which functions as a bearing that holds the treatment instrument operation lever 10 so as to be rotationally movable around the rotation axis Ax.

Note that the reference sign Z1 in FIG. 3 indicates the center axis of the first hole O1. The reference sign Z2 in FIG. 3 indicates the center axis of the second hole O2. In addition, the reference sign 51x in FIG. 3 indicates connecting arc portions that connect the first hole O1 and the second hole O2.

The two screw insertion slots 51d are formed respectively on outer edge portions of the two connecting arc portions 51x of the shaft insertion hole 51c. The screw insertion slots 51d are each formed in an arc shape along each of the connecting arc portions 51x. The screw insertion slots 51d are groove holes in which fastening screws (not shown) are inserted. The fastening screws are used for fixing the lever member 51 to the link main body 35 to be described later. In addition, the screw insertion slots 51d each function as a guide groove when the lever member 51 moves, as described later.

The treatment instrument operation lever 10 thus configured is arranged on an exterior surface of the operation portion 3 as shown in FIGS. 1-2, etc.

In this case, the treatment instrument operation lever 10 is, in the first state (normal use state), arranged at a position where the center axis Z1 of the first hole O1 of the lever member 51 coincides with the rotation axis Ax. At this time, the treatment instrument operation lever 10 is held, at the center axis Z1 of the first hole O1 of the lever member 51, so as to be rotationally movable around the rotation axis Ax.

In addition, the treatment instrument operation lever 10 is, in the second state (maintenance adaptive state), arranged at a position where the center axis Z2 of the second hole O2 of the lever member 51 coincides with the rotation axis Ax. At this time, the treatment instrument operation lever 10 is held, at the center axis Z2 of the second hole O2 of the lever member 51, so as to be rotationally movable around the rotation axis Ax.

When the treatment instrument operation lever 10 receives the operation input by the user, the treatment instrument operation lever 10 rotationally moves in a direction along the arrow R in FIG. 4 in each of the states. Thus, when the treatment instrument operation lever 10 rotationally moves, the workings of the driving mechanism 30 and the treatment instrument raising wire 27 cause a movable member (treatment instrument raising base 50), which is provided at the distal end portion 5, to move in a predetermined direction.

Note that the rotational movement range of the treatment instrument operation lever 10 in the direction of the arrow R is defined by a part of the finger contact member (not shown) of the treatment instrument operation lever 10 coming into contact with a predetermined wall surface of the operation portion 3. In this case, the wall surface of the operation portion 3 serves as a stopper that defines the normal operation range (rotational movement range) of the treatment instrument operation lever 10.

Next, description will be made briefly below on the configuration of the driving mechanism 30 of the treatment instrument raising base 50, with reference to FIG. 4.

The driving mechanism 30 in the endoscope 1 of the present embodiment is a link mechanism that receives the operation input through the treatment instrument operation lever 10, to convert the received operation input into a force for advancing and retreating the treatment instrument raising wire 27 in a longitudinal axis direction of the insertion portion 2. The treatment instrument raising wire 27 advanced and retreated through the driving mechanism 30 causes the treatment instrument raising base 50 to rotationally move in a predetermined direction, to raise the treatment instrument raising base 50.

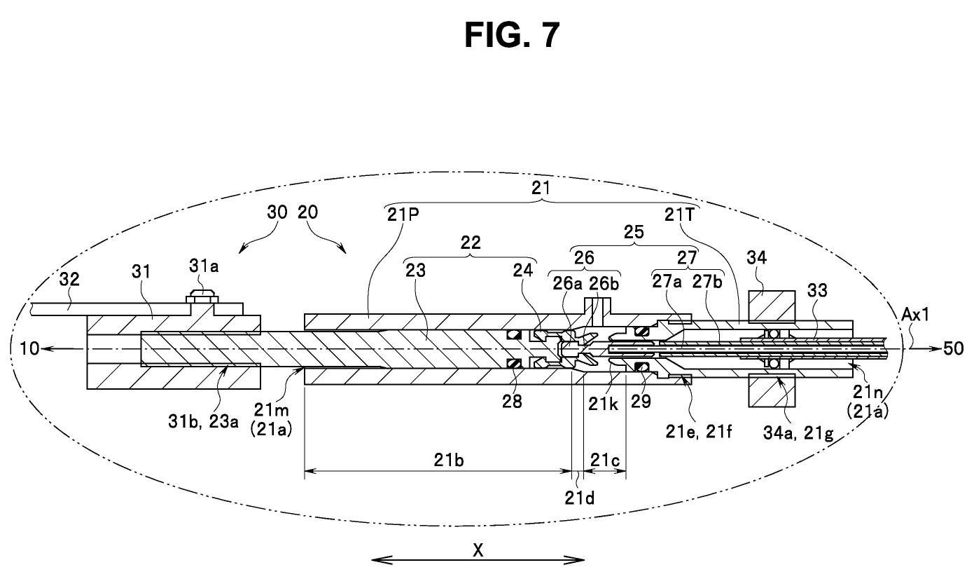

The driving mechanism 30 is a constituent unit attached and fixed to the inner fixing member (frame; not shown) of the operation portion 3. As shown in FIG. 4, the driving mechanism 30 includes the frame shaft 36, the link main body 35, and other constituent members (a lever connecting member 31, a link rod 32, etc.; see FIG. 7).

The frame shaft 36 is a shaft-like member standing in a substantially upright state with respect to a frame plane of the operation portion 3. The frame shaft 36 is fixed onto the frame plane by screw fastening, for example. Here, the frame shaft 36 is disposed such that the center axis thereof coincides with the rotation axis Ax which is the rotation center of the treatment instrument operation lever 10.

In addition, as shown in FIG. 4, the frame shaft 36 includes, on a part of an outer circumferential surface thereof, a cutout 36a. The frame shaft 36 is formed such that the cross section of the part where the cutout 36a is arranged, which is substantially orthogonal to the rotation axis Ax, is formed in a substantially D-shape.

As shown in FIG. 4, the link main body 35 is a member formed in a substantially annular shape as a whole and disposed so as to be rotationally movable around the frame shaft 36 (the direction of the arrow R in FIG. 4). To this end, the link main body 35 is formed by including, at substantially the center region thereof, a through hole 35a that is fitted to the frame shaft 36.

In addition, the link main body 35 includes, at a part of the outer peripheral edge portion thereof, a link portion 35b formed so as to protrude outwardly. To the link portion 35b, the proximal end of the link rod 32 shown in FIG. 7 is connected.

Furthermore, the link main body 35 includes, in the annular region thereof, a plurality of screw holes 35c. These screw holes 35c are the parts to which guiding shoulder screws 38a1 and 38a2 are respectively fastened. The guiding shoulder screws 38a1 and 38a2 are a plurality of fastening screws for fixing the lever member 51 of the treatment instrument operation lever 10.

The working of the treatment instrument operation lever 10 thus configured is as described below.

First, the treatment instrument operation lever 10 is assembled to the operation portion 3 as shown in FIGS. 4 and 5, in the normal use state.

In the normal use state (first state), the treatment instrument operation lever 10 (lever member 51) is assembled to the link main body 35 and the frame shaft 36 with the center axis Z1 of the first hole O1 made coincident with the rotation axis Ax, as shown in FIGS. 4 and 5.

With such a configuration, the treatment instrument operation lever 10 (lever member 51) is held at the center axis Z1 so as to be rotationally movable around the rotation axis Ax (the direction of the arrow R in FIG. 4). At this time, the lever member 51 is screw-fastened to the screw holes 35c of the link main body 35, at the respective one end portions of the two screw insertion slots 51d, using the guiding shoulder screws 38a1 and 38a2.

In other words, the lever member 51 and the link main body 35 are fastened with clearance being formed at the two screw insertion slots 51d by respectively using the guiding shoulder screws 38a1 and 38a2. With such a configuration, the lever member 51 is configured to be always movable with respect to the link main body 35 in the direction of the arrow R in FIG. 4 and in directions of the arrow M in FIGS. 5 and 6 (M1 and M2 directions).

Here, the screw indicated by the reference sign 38a1 penetrates the screw insertion slot 51d disposed on the inner side of the lever member 51. In addition, the screw indicated by the reference sign 38a2 penetrates the screw insertion slot 51d disposed on the outer side of the lever member 51.

At this time, when the lever member 51 receives the operation input and rotates in the direction of the arrow R1 shown in FIG. 5, the guiding shoulder screw 38a1 acts on the one end portion of the screw insertion slot 51d on the inner side, and thereby the lever member 51 causes the link main body 35 to rotationally move.

On the other hand, in the above-described state (the state in FIG. 4), if the treatment instrument operation lever 10 (lever member 51) is pulled in the direction of the arrow M1 in FIG. 5, the treatment instrument operation lever 10 (lever member 51) is brought into the state (second state) where the center axis Z2 of the second hole O2 coincides with the rotation axis Ax, as shown in FIG. 6.

At this time, when the lever member 51 receives the operation input and rotates in the direction of the arrow R1 in FIG. 6, the guiding shoulder screw 38a2 acts on the other end portion of the screw insertion slot 51d on the outer side, and thereby the lever member 51 causes the link main body 35 to rotationally move.

Note that, instead of the configuration using the guiding shoulder screws, the lever member 51 and the link main body 35 may be fastened with each other using slightly loosened screws.

As a further different configuration, for example, instead of the guiding shoulder screws, fastening screws of a normal type are used to fasten the lever member 51 and the link main body 35 with each other. Then, the fastening screws may be loosened each time when the lever member 51 is moved in the direction of the arrow M shown in FIGS. 5 and 6.

With such a configuration, the treatment instrument operation lever 10 (lever member 51) is held at the center axis Z2 so as to be rotationally movable around the rotation axis Ax. At this time, the lever member 51 is screwed to the link main body 35, at the respective other end portions of the two screw insertion slots 51d.

With the configuration as described above, the treatment instrument operation lever 10 is configured such that it can be changed between the state (first state) where the center axis Z1 of the first hole O1 coincides with the rotation axis Ax and the state (second state) where the center axis Z2 of the second hole O2 coincides with the rotation axis Ax.

The treatment instrument operation lever 10 is configured such that, when rotationally moved in the second state, the finger contact member (not shown) does not come into contact with the wall surface of the operation portion 3. Such a configuration allows, in the second state, the treatment instrument operation lever 10 to rotationally move to the maintenance operation range which is the rotational movement range exceeding the normal operation range.

Then, when the treatment instrument operation lever 10 is in the first state (the state in FIG. 5; normal operation range), a first connecting member (a chuck 24) is positioned in a first part (small-diameter portion 21b). In addition, when the treatment instrument operation lever 10 is in the second state and in the maintenance operation range, the first connecting member (chuck 24) is positioned in a second part (large-diameter portion 21c).

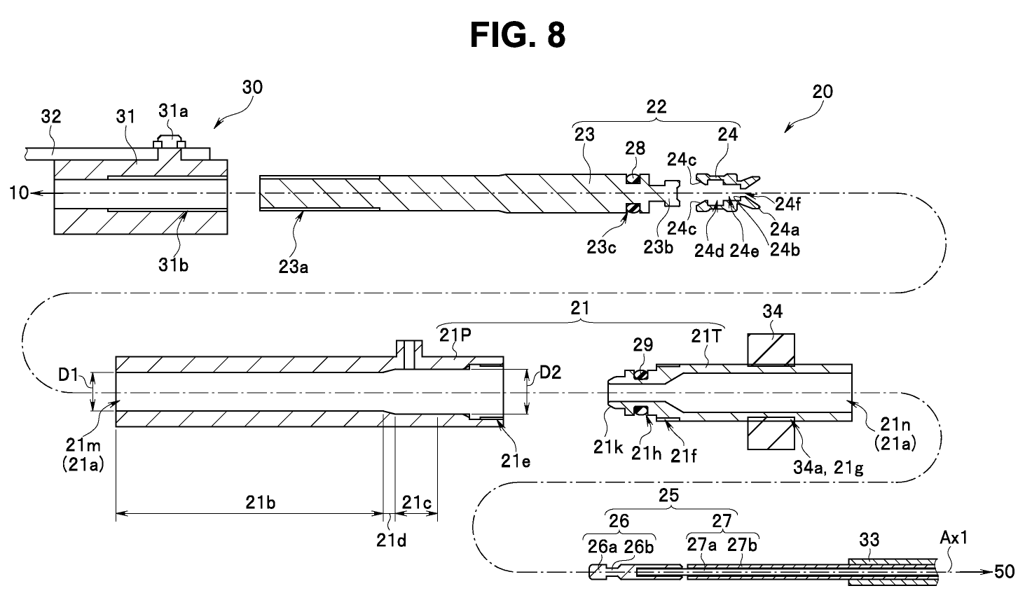

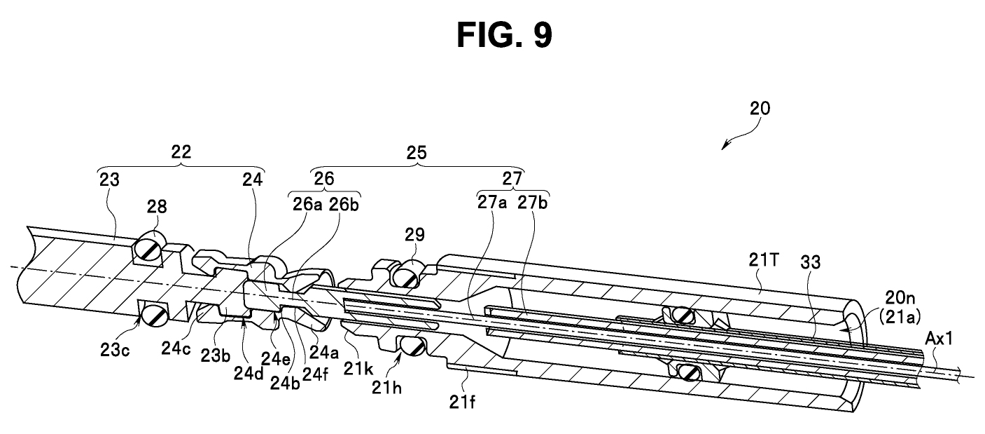

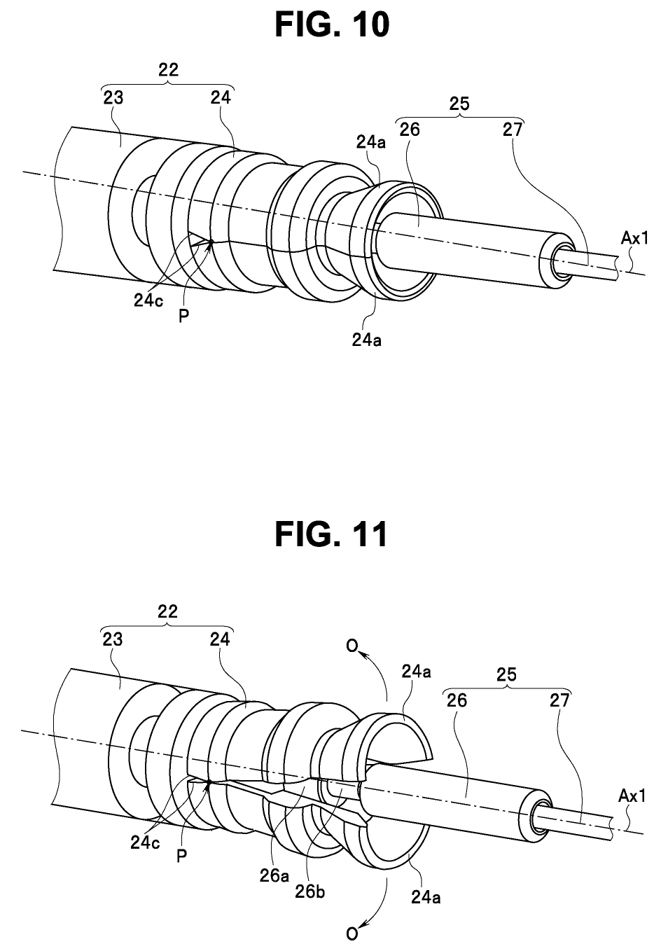

Next, description will be made on a detailed configuration of the treatment instrument raising wire connection mechanism 20 in the endoscope 1 in the first embodiment of the present disclosure, with reference to FIGS. 7 to 11. FIG. 7 shows the configuration of the treatment instrument raising wire connection mechanism in the endoscope in FIG. 1. FIG. 7 is a main-part enlarged cross-sectional view showing, in an enlarged manner, a region indicated by the reference sign [7] in FIG. 2. FIG. 8 shows cross sections of the respective constituent members in a state where the treatment instrument raising wire connection mechanism shown in FIG. 7 is disassembled. FIG. 9 is a cross-sectional perspective view of the treatment instrument raising wire connection mechanism shown in FIG. 7, which is partly enlarged and viewed in an oblique direction. Note that, in FIG. 9, some of the constituent members (a part of a cylinder, etc.) among the respective constituent members of the treatment instrument raising wire connection mechanism are omitted. FIG. 10 and FIG. 11 are each a main-part enlarged perspective view showing the first connecting member and the second connecting member in the treatment instrument raising wire connection mechanism in FIG. 7. Of these figures, FIG. 10 shows a state where the first connecting member is closed. In addition, FIG. 11 shows a state where the first connecting member is open.

The treatment instrument raising wire connection mechanism 20 in the endoscope of the present embodiment is provided between the driving mechanism 30 including the treatment instrument operation lever 10 and the treatment instrument raising base 50, and is a mechanism for performing connection and separation of the two (30, 50) appropriately at a desired time.

Since the treatment instrument raising wire connection mechanism 20 is provided, the driving mechanism 30 and the treatment instrument raising base 50 can be surely connected to each other when the endoscope is in use. Therefore, in this state, the raising and lowering operation and the like of the treatment instrument raising base 50 can be surely performed by using the treatment instrument operation lever 10.

On the other hand, when the endoscope is not in use, and for example, at the time of the maintenance or cleaning of the endoscope, the treatment instrument raising wire connection mechanism 20 enables easy separation between the driving mechanism 30 and the treatment instrument raising base 50 by a simple operation. Therefore, in this case, the predetermined work such as the replacement work of the treatment instrument raising wire 27 can be performed easily.

To this end, the treatment instrument raising wire connection mechanism 20 in the endoscope 1 in the present embodiment is provided in the operation portion 3, as shown in FIGS. 7-8, etc. The treatment instrument raising wire connection mechanism 20 includes a cylinder 21, a first moving member 22, and a second moving member 25, and an opening/closing mechanism.

The cylinder 21 includes a first cylinder 21P and a second cylinder 21T. The first cylinder 21P and the second cylinder 21T are each formed in a substantially hollow cylindrical shape as a whole.

The first cylinder 21P is a member configured such that a part of the first moving member 22 and a part of the second moving member 25 are inserted and arranged therein, to thereby guide the movement of the first moving member 22 and the second moving member 25 in a predetermined direction. For this purpose, the first cylinder 21P includes a first inner space 21m.

The second cylinder 21T is a member configured such that a part of the second moving member 25 is inserted and arranged therein, to thereby guide the movement of the second moving member 25 in the predetermined direction. In addition, the second cylinder 21T is configured such that a part of the treatment instrument raising wire insertion conduit 33 (see FIG. 7, etc.) is inserted therein, to fix and hold the proximal end of the treatment instrument raising wire insertion conduit 33. For this purpose, the second cylinder 21T includes a second inner space 21n.

The first cylinder 21P and the second cylinder 21T are connected integrally with each other. In addition, in the state where the first cylinder 21P and the second cylinder 21T are integrated with each other, the second cylinder 21T is fixed to a fixing portion 34 in the operation portion 3.

Therefore, the first cylinder 21P includes, on the inner circumferential surface at the distal end thereof, a screw portion 21e. The second cylinder 21T includes, on the outer circumferential surface at the side closer to the proximal end thereof, a screw portion 21f formed so as to correspond to the screw portion 21e. Then, the screw portion 21e and the screw portion 21f are screwed together, and thereby the first cylinder 21P and the second cylinder 21T are connected integrally with each other.

Furthermore, the second cylinder 21T is screw-fixed to the fixing portion 34 in the operation portion 3. To this end, a first adjusting screw portion 21g is formed on the outer circumferential surface of the second cylinder 21T so as to be located at a position in the middle of the second cylinder 21T. The fixing portion 34 includes a screw portion 34a formed so as to correspond to the first adjusting screw portion 21g. Then, the first adjusting screw portion 21g and the screw portion 34a are screwed together, and thereby the second cylinder 21T is fixed to the fixing portion 34 of the operation portion 3.

In this case, the screwed state between the first adjusting screw portion 21g and the screw portion 34a is controlled, to thereby be capable of adjusting the relative position of the second cylinder 21T with respect to the fixing portion 34. Thus, the position adjustment of the cylinder 21 in the operation portion 3 can be performed.

Note that, at a part closer to the distal end of the second cylinder 21T, a circumferential groove 21h (see FIG. 8 and FIG. 9) is formed along the outer circumferential surface of the second cylinder 21T. The circumferential groove 21h accommodates an O-ring 29 therein. The O-ring 29 is provided for sealing between the inner circumferential surface of the first cylinder 21P and the outer circumferential surface of the second cylinder 21T.

When the first cylinder 21P and the second cylinder 21T are connected integrally, the cylinder 21 includes an inner space 21a in which the first inner space 21m and the second inner space 21n are communicated with each other. The inner space 21a is formed along a first axis Ax1 extending from the distal end toward the proximal end. In addition, the inner space 21a penetrates the cylinder 21 in a direction along the first axis Ax1.

As described above, a part of the first moving member 22 and a part of the second moving member 25 are inserted and arranged in the inner space 21a. At this time, the first moving member 22 and the second moving member 25 are disposed so as to be movable in the inner space 21a in the direction of the arrow X in FIG. 7, etc., along the first axis Ax1.

The first moving member 22 includes a piston rod 23 and the chuck 24 as the first connecting member. The piston rod 23 is a rod member formed in a columnar shape as a whole. The proximal end of the piston rod 23 is fixed to the driving mechanism 30. Therefore, the piston rod 23 includes, on the outer circumferential surface on the proximal end thereof, a second adjusting screw portion 23a. The lever connecting member 31 (to be described later) of the driving mechanism 30 includes a screw portion 31b formed so as to correspond to the second adjusting screw portion 23a. Then, the second adjusting screw portion 23a and the screw portion 31b are screwed together, and thereby the piston rod 23 is fixed to the driving mechanism 30. In this case, the screwed state between the second adjusting screw portion 23a and the screw portion 31b is controlled, to thereby be capable of adjusting the relative position between the piston rod 23 and the driving mechanism 30.

The piston rod 23 includes, at the distal end thereof, the chuck 24. Therefore, the piston rod 23 includes, at the distal end thereof, a flange-shaped engaged portion 23b (see FIG. 8 and FIG. 9). The engaged portion 23b is, as will be described later, a part that is engaged by an engaging portion 24d of the chuck 24. Then, the engaged portion 23b is engaged by the engaging portion 24d of the chuck 24, thereby enabling the piston rod 23 and the chuck 24 to be integrated.

Note that, at a part closer to the distal end of the piston rod 23, a circumferential groove 23c is formed along the outer circumferential surface of the piston rod 23. The circumferential groove 23c includes an O-ring 28 therein. The O-ring 28 is provided for sealing between the inner circumferential surface of the cylinder 21 and the outer circumferential surface of the piston rod 23.

As shown in FIGS. 10-11, etc., the chuck 24 is formed in a substantially cylindrical shape as a whole, and is the first connecting member formed by including a distal-end tapered portion 24a, claw portions 24b, a cutout portion 24c, the engaging portion 24d, a holding portion 24e, and a through hole 24f. Note that the chuck 24 is formed by using a material such as a metal.

The chuck 24 shown in the present embodiment is formed so as to have two segments by dividing a substantially cylindrical member into two along a plane including the first axis Ax1.

The state shown in FIG. 10 shows a state where the two members formed as the two segments of the chuck 24 are combined at a seam along the first axis Ax1. The state shown in FIG. 10 is referred to as a closed state of the chuck 24.

In addition, the state shown in FIG. 11 shows a state where the two members formed as the two segments of the chuck 24 are separated from each other at the seam along the first axis Ax1. The state shown in FIG. 11 is referred to as an open state of the chuck 24.

The distal-end tapered portion 24a is formed at the distal end of the chuck 24. The distal-end tapered portion 24a is formed in a tapered shape such that the inner surface thereof expands toward the distal end side. The distal-end tapered portion 24a is configured such that, when the chuck 24 is in the closed state (see FIG. 9 and FIG. 10), the through hole 24f is defined at the center portion of the proximal end part of the distal-end tapered portion 24a, along the first axis Ax1. When the chuck 24 is in the closed state, a neck portion 26b of a second connecting member 26 to be described later is engaged in the through hole 24f.

The claw portions 24b are each a part formed at the proximal end part of the distal-end tapered portion 24a, and has a cross section having a shape protruding in the radial direction of the first axis Ax1. The claw portions 24b are formed so as to catch and hold one part of the second moving member 25 (a head portion 26a of the second connecting member 26; to be described later), when the chuck 24 is in the closed state. In the present embodiment, since the chuck 24 is formed of the two members, the chuck 24 includes at least two claw portions 24b.

When the chuck 24 is in the closed state, and the claw portions 24b catch and hold the one part (head portion 26a; to be described later) of the second moving member 25, the holding portion 24e has a space for housing the one part (head portion 26a) of the second moving member 25. The holding portion 24e is a part opposed to the claw portions 24b of the chuck 24, and is formed at a position closer to the proximal end than the claw portions 24b.

The engaging portion 24d is a part with which the engaged portion 23b of the piston rod 23 is engaged, to thereby allow the chuck 24 to be disposed integrally with the distal end of the piston rod 23. The engaging portion 24d is a part adjacent to the holding portion 24e and formed at a part closer to the proximal end than the holding portion 24e.

The cutout portion 24c is formed at the proximal end of the chuck 24. When the two members, which are formed as the two segments, of the chuck 24 are in the open state (the state shown in FIG. 11), the two members of the chuck 24 separate from each other such that each of the two members rotationally moves with the part indicated by the reference sign P in FIG. 11 as a rotational movement center, along the reference sign O in FIG. 11 (becomes the open state). The cutout portion 24c is formed to allow the rotational movement of the chuck 24.

Note that, as also understood from the above description, the chuck 24 as the first connecting member is positioned at a side closer to the distal end than the bending operation knob 9 (angle knob) and the driving mechanism 30.

The second moving member 25 includes the second connecting member 26 and the treatment instrument raising wire 27.

The second connecting member 26 is a member provided for connecting the first moving member 22 and the second moving member 25 by the one part (head portion 26a) on the proximal end side of the second connecting member 26 being held by the holding portion 24e of the chuck 24 (first connecting member).

The second connecting member 26 includes the head portion 26a and the neck portion 26b. When the chuck 24 is in the closed state, the head portion 26a is housed in the holding portion 24e of the chuck 24. At this time, the two claw portions 24b catch and hold the head portion 26a.

The neck portion 26b is connected to the distal end side of the head portion 26a and formed to be narrower than the head portion 26a. When the chuck 24 is brought into the closed state with the head portion 26a being held by the holding portion 24e of the chuck 24, the neck portion 26b is engaged in the through hole 24f at the proximal end part of the distal-end tapered portion 24a. With such a configuration, the head portion 26a is brought into a state where removal from the holding portion 24e is not allowed easily. Therefore, in this state, the head portion 26a is surely held by the two claw portions 24b and the holding portion 24e.

The treatment instrument raising wire 27 includes a wire 27a and a covering tube 27b. The wire 27a is fixed to the distal end of the second connecting member 26. The wire 27a is formed of a flexible elongated linear member made of metal, for example. The outer circumferential side of the wire 27a is covered with the covering tube 27b. The covering tube 27b is formed of a heat-shrinkable tube, a coil, and the like, for example.

The reason why the treatment instrument raising wire 27 is configured such that the wire 27a is covered with the covering tube 27b will be described below.

In general, if the wire 27a alone, for example, is inserted into the treatment instrument raising wire insertion conduit 33, a gap is created between the inner surface of the treatment instrument raising wire insertion conduit 33 and the outer surface of the wire 27a. If such a gap exists in the treatment instrument raising wire insertion conduit 33, when the wire 27a advances or retreats in the treatment instrument raising wire insertion conduit 33, meandering and the like occur in the wire 27a in some cases. If such meandering and the like occur, the raising and lowering operation of the treatment instrument raising base 50, which works in accordance with the advancing and retreating movement of the wire 27a, is possibly hindered due to the meandering and the like.

In view of the above, the present embodiment employs the treatment instrument raising wire 27 configured such that the wire 27a is covered with the covering tube 27b. In such a configuration, when the treatment instrument raising wire 27 is inserted in the treatment instrument raising wire insertion conduit 33, the gap between the inner surface of the treatment instrument raising wire insertion conduit 33 and the outer surface of the treatment instrument raising wire 27 can be filled up. Therefore, with such a configuration, meandering and the like do not occur in the treatment instrument raising wire 27 in the treatment instrument raising wire insertion conduit 33, and sure advancing and retreating operation can be ensured.

As described above, the treatment instrument raising wire 27 is inserted into the treatment instrument raising wire insertion conduit 33 (see FIG. 7, etc.), which is inserted and arranged in the insertion portion 2 and the operation portion 3, and advanceable and retreatable in the direction (direction of the arrow X) along the longitudinal axis (first axis Ax1) of the treatment instrument raising wire insertion conduit 33.

The opening/closing mechanism is configured to change the state of the chuck 24 between the state where the chuck 24 is closed to hold the second connecting member 26 (see FIG. 10) and the state where the chuck 24 is opened to release the second connecting member 26 (see FIG. 11).

The opening/closing mechanism of the treatment instrument raising wire connection mechanism 20 shown in the present embodiment is formed in at least a part of the cylinder 21. In other words, the opening/closing mechanism forms at least a part of the cylinder 21. Namely, the opening/closing mechanism is, for example, a part of the inner space 21a of the cylinder 21, and formed in a part of the first inner space 21m of the first cylinder 21P.

In detail, as shown in FIG. 7, etc., in the first inner space 21m of the first cylinder 21P, the small-diameter portion 21b which is the first part, and the large-diameter portion 21c which is the second part, and a tapered portion 21d which is a third part are formed.

The small-diameter portion 21b has a first cross-sectional area. The first cross-sectional area can be calculated based on a diameter indicated by the reference sign D1 in FIG. 8, when the cross-sectional shape of the small-diameter portion 21b of the first inner space 21m has a circular shape, for example. The first cross-sectional area of the small-diameter portion 21b is set to a size that does not allow for the chuck 24 to release the second connecting member 26.

Accordingly, when the chuck 24, which is in the closed state with the head portion 26a of the second connecting member 26 being held by the holding portion 24e of the chuck 24, is arranged in the small-diameter portion 21b, the chuck 24 does not allow the second connecting member 26 to be released. In other words, when the chuck 24 is in this state, the chuck 24 is restricted from being in the open state by the opening/closing mechanism.

The large-diameter portion 21c is provided on the distal end side of the small-diameter portion 21b. The large-diameter portion 21c has a second cross-sectional area larger than the first cross-sectional area. The second cross-sectional area can be calculated based on a diameter indicated by the reference sign D2 in FIG. 8, when the cross-sectional shape of the large-diameter portion 21c of the first inner space 21m has a circular shape, for example. The second cross-sectional area of the large-diameter portion 21c is set to a size that allows for the chuck 24 to release the second connecting member 26.

Accordingly, when the chuck 24, which is in the closed state with the head portion 26a of the second connecting member 26 being held by the holding portion 24e of the chuck 24, is arranged in the large-diameter portion 21c, the chuck 24 allows the second connecting member 26 to be released. In other words, at this time, the chuck 24 can be in the open state.

The tapered portion 21d is a part connecting the small-diameter portion 21b and the large-diameter portion 21c. The tapered portion 21d is formed in a tapered shape expanding from the distal end of the small-diameter portion 21b toward the distal end side, that is, the side where the large-diameter portion 21c is located.

With such a configuration, in the case where the chuck 24 moves from the large-diameter portion 21c to the small-diameter portion 21b in the direction along the first axis Ax1, when the distal-end tapered portion 24a of the chuck 24 passes through the tapered portion 21d of the first cylinder 21P, the chuck 24, which is in the open state, moves with the outer surface of the distal-end tapered portion 24a contacting the inner surface of the tapered portion 21d. This movement allows smooth closing of the chuck 24.

The cylinder 21 further includes a fourth part. Specifically, the fourth part is a receiving portion 21k provided at the proximal end of the second cylinder 21T. The receiving portion 21k is disposed in the large-diameter portion 21c. The receiving portion 21k is formed in a tapered shape expanding toward the distal end side.

With such a configuration, when the chuck 24 moves from the small-diameter portion 21b to the large-diameter portion 21c in the direction along the first axis Ax1, the chuck 24 moves with the inner surface of the distal-end tapered portion 24a of the chuck 24 contacting the receiving portion 21k. This movement allows smooth opening of the chuck 24.

Meanwhile, the driving mechanism 30 is a constituent unit configured to transmit an operation force of the treatment instrument operation lever 10 to the treatment instrument raising base 50 through the treatment instrument raising wire connection mechanism 20.

As shown in FIG. 7, etc., the driving mechanism 30 includes the lever connecting member 31, the link mechanism (not shown) including the link rod 32, the treatment instrument operation lever 10 (not shown in FIG. 7; see FIG. 1, etc.), and the like.

For example, the distal end of the link rod 32 is connected to a link coupling shaft 31a of the driving mechanism 30. The proximal end of the link rod 32 is connected to a predetermined link mechanism (not shown). To the link mechanism, the treatment instrument operation lever 10 is connected.

In addition, as described above, the proximal end (second adjusting screw portion 23a) of the piston rod 23 is connected to the distal end (screw portion 31b) of the lever connecting member 31.

With such a configuration, when the treatment instrument operation lever 10 is operated to be rotationally moved in a predetermined direction, the lever connecting member 31 and the piston rod 23 move in the direction along the first axis Ax1 (direction of the arrow X). When the piston rod 23 (first moving member 22) moves in the direction of the arrow X, also the second moving member 25 moves in the same direction. With this, the treatment instrument raising wire 27 causes the treatment instrument raising base 50 to rise or fall.

Note that the configurations of the treatment instrument raising base 50, the driving mechanism 30, the treatment instrument operation lever 10, and the like are substantially the same as the corresponding configurations in the conventional endoscopes. Therefore, illustration and description of the detailed configurations are omitted.

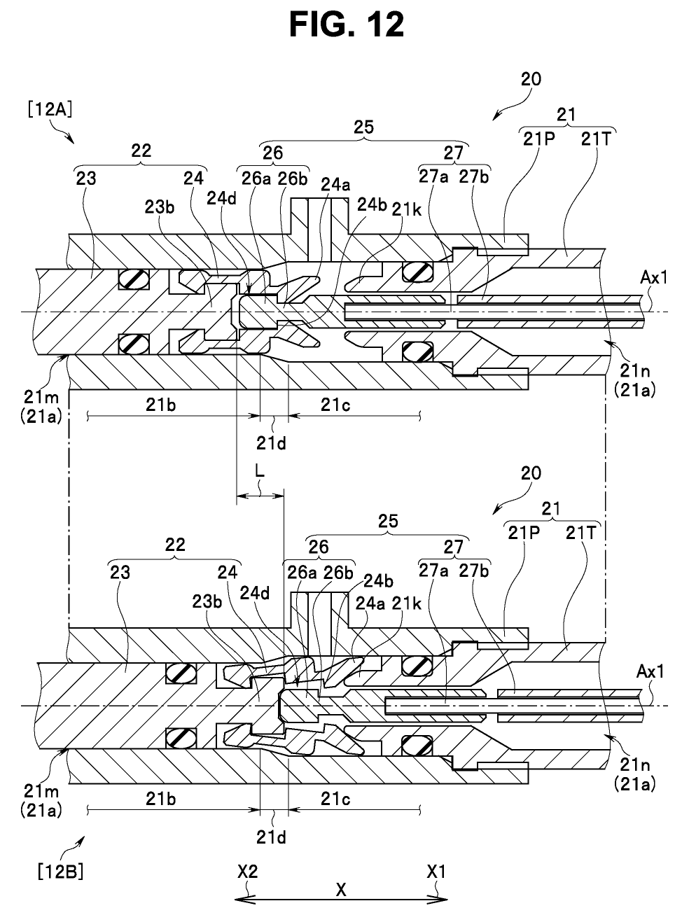

The working of the treatment instrument raising wire connection mechanism 20 in the endoscope 1 in the first embodiment of the present disclosure configured as described above, will be described with reference to FIG. 12. FIG. 12 shows the working of the treatment instrument raising wire connection mechanism in the endoscope in the first embodiment of the present disclosure.

First, it is supposed that the treatment instrument raising wire connection mechanism 20 is in the state indicated by the reference sign [12A] in FIG. 12. In this state, the head portion 26a of the second connecting member 26 is held by the holding portion 24e of the chuck 24, and the chuck 24 is closed. In other words, in this state, the first moving member 22 (chuck 24) and the second moving member 25 (second connecting member 26) are connected to each other.

In addition, in this state, the proximal end side part, which includes at least the holding portion 24e, of the chuck 24 is arranged in the small-diameter portion 21b of the first inner space 21m. With this, the chuck 24 is restricted from being in the open state.

Then, in this state, the treatment instrument raising wire connection mechanism 20 surely connects the first moving member 22 and the second moving member 25. In other words, in this state, the operation force of the treatment instrument operation lever 10 can be transmitted to the treatment instrument raising base 50 through the driving mechanism 30, the treatment instrument raising wire connection mechanism 20, and the treatment instrument raising wire 27.

Accordingly, when the treatment instrument raising wire connection mechanism 20 is in the connected state indicated by the reference sign [12A] in FIG. 12, if the treatment instrument operation lever 10 is operated voluntarily within a predetermined normal operation range, the raising and lowering operation of the treatment instrument raising base 50 can be performed. In other words, when the endoscope 1 is in use, the treatment instrument raising wire connection mechanism 20 is in the state indicated by the reference sign [12A] in FIG. 12.

Thus, when the treatment instrument raising wire connection mechanism 20 is in the connected state indicated by the reference sign [12A] in FIG. 12, the treatment instrument operation lever 10 is normally set to operate within the normal operation range. For example, when the maintenance work of the endoscope 1 such as the replacement work of the treatment instrument raising wire 27 is performed, a predetermined operation is performed on the treatment instrument operation lever 10, to switch the treatment instrument operation lever 10 to the maintenance operation range setting that allows the treatment instrument operation lever 10 to move to the maintenance operation range.

In this state, the treatment instrument operation lever 10 is operated in a predetermined direction. Further, the treatment instrument operation lever 10 is rotationally moved to the maintenance operation range that exceeds the normal operation range. Then, in this state, the operation force of the treatment instrument operation lever 10 acts on the first moving member 22 of the treatment instrument raising wire connection mechanism 20 through the driving mechanism 30.

With this, it is supposed that the first moving member 22 has moved, in the direction along the first axis Ax1, for example, toward the direction of the arrow X1 in FIG. 12 by a predetermined distance L. As shown in FIG. 12, the predetermined distance L indicates the distance in the case where the distal end surface of the piston rod 23 has moved from the position indicated by the reference sign [12A] in FIG. 12 to the position indicated by the reference sign [12B] in FIG. 12.

In this case, the chuck 24 also moves in the direction of the arrow X1 by the same distance. Such a movement of the chuck 24 causes the distal-end tapered portion 24a, which has been arranged in the small-diameter portion 21b in the state of the reference sign [12A] in FIG. 12, to move to a position where the distal-end tapered portion 24a is arranged in the large-diameter portion 21c.

According to the movement of the chuck 24 in the direction of the arrow X1, the distal end of the receiving portion 21k comes into contact with the inner surface of the distal-end tapered portion 24a of the chuck 24. When the chuck 24 is further moved to the direction of the arrow X1 in this state, the inner surface of the distal-end tapered portion 24a is pressed against the receiving portion 21k, to thereby cause the distal-end tapered portion 24a to open. At the same time, the holding portion 24e moves, while the outer surface thereof coming into contact with the inner surface of the tapered portion 21d. This causes the distal-end tapered portion 24a of the chuck 24 to gradually change to the open state.

Thus, when the chuck 24 is brought into the state (the state indicated by the reference sign [12B] in FIG. 12) where the chuck 24 is arranged in the large-diameter portion 21c, at the same time, the chuck 24 is brought into the open state by the receiving portion 21k. Then, the chuck 24 is brought into the state that allows the head portion 26a of the second connecting member 26 to be released.

When the treatment instrument raising wire 27 is pulled toward the distal end side (in the direction along the arrow X1), for example, the head portion 26a of the second connecting member 26 is removed from the holding portion 24e, and thereby the connection between the first moving member 22 and the second moving member 25 is released. Thus, the treatment instrument raising wire 27 becomes a removable state.

After the treatment instrument raising wire 27 is pulled (toward the direction along the arrow X1), cleaning, etc., of the endoscope 1 is performed, for example. After that, a new treatment instrument raising wire 27 (second moving member 25) with the second connecting member 26 being attached to the proximal end thereof is inserted into the treatment instrument raising wire insertion conduit 33.

After the second moving member 25 inserted in the treatment instrument raising wire insertion conduit 33 reaches the treatment instrument raising wire connection mechanism 20, when the second moving member 25 is further pushed (toward the direction along the arrow X2), the head portion 26a of the second connecting member 26 moves while coming into contact with the inner surface of the distal-end tapered portion 24a of the chuck 24. Then, the head portion 26a opens the distal-end tapered portion 24a, and thereafter passes through the through hole 24f to enter the holding portion 24e.

In this state, when the treatment instrument operation lever 10 is operated in the predetermined direction, the operation force of the treatment instrument operation lever 10 causes the first moving member 22 to move in the direction of the arrow X2 in FIG. 12. This causes the chuck 24 to move from the large-diameter portion 21c to the small-diameter portion 21b via the tapered portion 21d. At this time, the chuck 24 moves with the outer surface of the holding portion 24e contacting the inner surface of the tapered portion 21d. With this, the chuck 24 smoothly changes to the closed state.

When the chuck 24 is arranged in the small-diameter portion 21b, to be brought into the state indicated by the reference sign [12A] in FIG. 12, the chuck 24 is restricted from being in the open state.

Meanwhile, at the time of manufacturing the endoscope 1, a procedure of attaching the treatment instrument raising wire is substantially as described below.

First, suppose, for instance, a state, as indicated by the reference sign [12A] or [12B] in FIG. 12, where the second moving member 25 (the second connecting member 26 and the treatment instrument raising wire 27) is not attached to the inside of the endoscope 1.

In this state, a predetermined operation is performed on the treatment instrument operation lever 10, to cause the first moving member 22 to move in the direction which is along the first axis Ax1 and which is the direction of the arrow X1 in FIG. 12. Simultaneously, the chuck 24 moves from the small-diameter portion 21b to the large-diameter portion 21c. When the chuck 24 is arranged in the large-diameter portion 21c, the chuck 24 is allowed to be in the open state.

Subsequently, when the chuck 24 is moved in the direction of the arrow X1, the distal-end tapered portion 24a of the chuck 24 comes into contact with the receiving portion 21k of the second cylinder 21T. In this state, when the chuck 24 is further moved in the direction of the arrow X1, the distal-end tapered portion 24a is pressed against the receiving portion 21k, to cause the chuck 24 to be in the open state.

The second moving member 25 is inserted into the treatment instrument raising wire insertion conduit 33. In this case, the second moving member 25 is advanced from the distal end portion 5 side in the direction which is along the first axis Ax1 and which is the direction of the arrow X2.

The head portion 26a of the second connecting member 26 of the second moving member 25 reaches the treatment instrument raising wire connection mechanism 20. Then, the head portion 26a comes into contact with the distal-end tapered portion 24a of the chuck 24. When the second moving member 25 is further advanced from this state in the same direction (direction of the arrow X2), the head portion 26a passes from the distal-end tapered portion 24a through the through hole 24f to enter the holding portion 24e.

When the second moving member 25 is further advanced from this state in the same direction (direction of the arrow X2), the head portion 26a comes into contact with the distal end of the engaged portion 23b of the piston rod 23, to press the piston rod 23 in the same direction (the direction of the arrow X2). At the same time, the chuck 24 moves in the same direction (the direction of the arrow X2).

When the chuck 24 moves in the direction of the arrow X2, the outer surface of the holding portion 24e of the chuck 24 contacts the inner surface of the tapered portion 21d of the first cylinder 21P, and moves in the same direction (direction of the arrow X2) along the inner surface. With this, the chuck 24 is gradually changed to the closing state. Then, the head portion 26a is housed in the holding portion 24e. In addition, a part of the head portion 26a is brought into the state caught by the two claw portions 24b.

The first moving member 22 is pushed by the head portion 26a, to cause the holding portion 24e of the chuck 24 to be arranged in the small-diameter portion 21b. This causes the chuck 24 to be in the closed state. Then, the first connecting member (chuck 24) and the second connecting member 26 are in the connected state.

As described above, according to the first embodiment, in the endoscope 1 provided with the treatment instrument raising base 50, the treatment instrument raising wire connection mechanism 20 including the treatment instrument raising wire 27 is provided between the driving mechanism 30 including the treatment instrument operation lever 10 and the treatment instrument raising base 50. In this case, the treatment instrument raising wire connection mechanism 20 includes the cylinder 21, the first moving member 22, the second moving member 25, and the opening/closing mechanism.

The cylinder 21 includes the inner space 21a formed along the first axis Ax1 extending from the distal end toward the proximal end. The first moving member 22 and the second moving member 25 are movable in the inner space 21a along the first axis Ax1.

The first moving member 22 includes the first connecting member (chuck 24) having at least two claw portions 24b configured to catch and hold a part (the head portion 26a of the second connecting member 26) of the second moving member 25.

The second moving member 25 includes the head portion 26a configured to be held by the first connecting member (chuck 24), and the second connecting member 26 including the neck portion 26b provided on the distal end side of the head portion 26a and formed to be narrower than the head portion 26a.

The opening/closing mechanism is configured to change the state of the first connecting member (the chuck 24) between the state where the first connecting member (chuck 24) is opened to release (the head portion 26a of) the second connecting member 26 and the state where the first connecting member (the chuck 24) is closed to hold (the head portion 26a of) the second connecting member 26.

The opening/closing mechanism is formed in at least a part of the inner space 21a of the cylinder 21. In other words, the inner space 21a includes the first part (small-diameter portion 21b) having the first cross-sectional area and the second part (large-diameter portion 21c) having the second cross-sectional area larger than the first cross-sectional area. Namely, the first part (small-diameter portion 21b) and the second part (large-diameter portion 21c) form at least a part of the inner space 21a. The second part (large-diameter portion 21c) is provided on the distal end side of the first part (small-diameter portion 21b).

The first cross-sectional area is set to the size that does not allow for the first connecting member (the chuck 24) to release (the head portion 26a of) the second connecting member 26. The second cross-sectional area is set to the size that allows for the first connecting member (the chuck 24) to release (the head portion 26a of) the second connecting member 26.

In addition, when the inner space 21a is formed by further including the third part (tapered portion 21d), the third part (tapered portion 21d) forms a tapered portion configured to connect the first part (small-diameter portion 21b) and the second part (large-diameter portion 21c). The tapered portion contacts the first connecting member (chuck 24) to close the first connecting member (chuck 24).

The cylinder 21 includes the fourth part (receiving portion 21k). The fourth part (receiving portion 21k) includes a receiving portion provided in the second part (large-diameter portion 21c) and formed in the shape expanding toward the distal end side. The receiving portion 21k contacts the distal-end tapered portion 24a of the first connecting member (chuck 24), to open the first connecting member (chuck 24).

The treatment instrument raising wire connection mechanism 20 thus configured is capable of surely ensuring the connected state between the first moving member 22 and the second moving member 25 when the endoscope is in use, regardless of a simple configuration with reduced number of components. On the other hand, at the time of maintenance, and the like of the endoscope, the connected state of the first moving member 22 and the second moving member 25 can be easily released, which enables simplification of the attaching/detaching work of the treatment instrument raising wire 27, for example.

Note that, in the above-described first embodiment, the configuration has been described in which all of the first part (small-diameter portion 21b), the second part (large-diameter portion 21c), the third part (tapered portion 21d), and the fourth part (receiving portion 21k) are included as the components of the opening/closing mechanism. However, the configuration is not limited to the above-described one.

For example, the opening/closing mechanism may be configured by excluding the third part and the fourth part from the above-described first embodiment. In this case, a step is formed at the connected part between the first part and the second part. Therefore, in this case, when the chuck 24 moves in the inner space 21a from the second part to the first part, the chuck 24 is suddenly opened or closed in the step of the connected part between the first part and the second part.

In contrast, in the case where the opening/closing mechanism is configured by including the third part, when the chuck 24 moves from the second part to the first part, the chuck 24 moves along the third part. This allows the smooth closing of the chuck 24.

Furthermore, in the case where the opening/closing mechanism is configured by additionally including the fourth part, when the chuck 24 moves from the first part to the second part, the inner surface of the distal-end tapered portion 24a of the chuck 24 contacts the receiving portion 21k to be pressed against the receiving portion 21k. This allows the chuck 24 to be changed smoothly to the open state.

Meanwhile, in the present embodiment, the example is shown in which the chuck 24 is provided on the first moving member 22 (piston) side. However, the configuration is not limited to the example. For example, the chuck 24 may be provided on the second moving member 25 side.

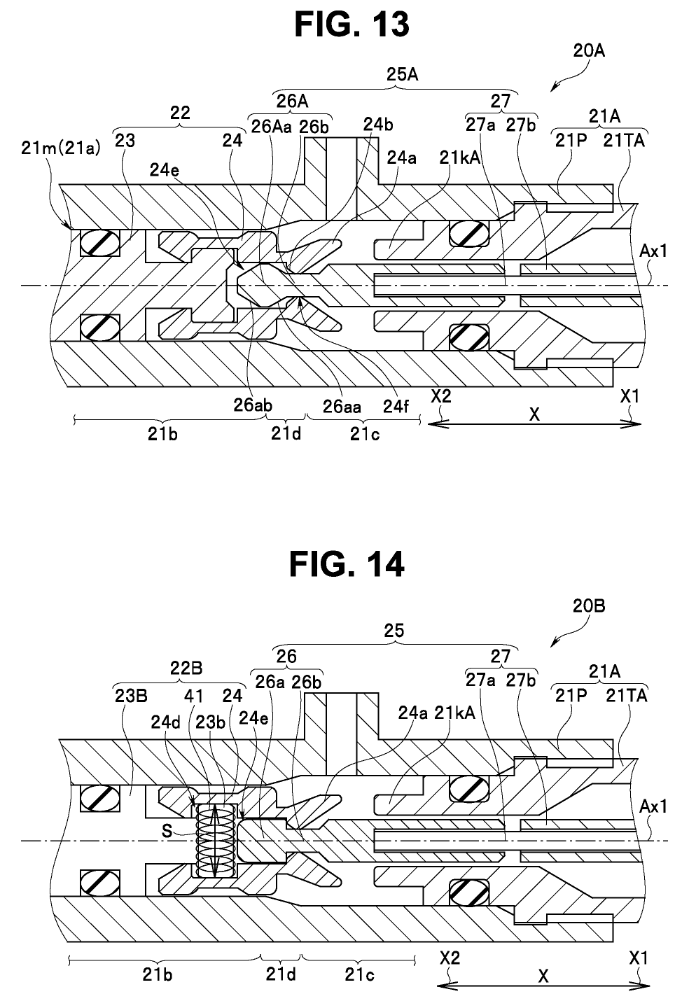

Next, a second embodiment of the present disclosure will be described below. FIG. 13 is a main-part enlarged cross-sectional view showing, in an enlarged manner, a main part (connecting part between a first connecting member and a second connecting member) of a treatment instrument raising wire connection mechanism in an endoscope in the second embodiment of the present disclosure. Note that FIG. 13 shows a state where the first connecting member and the second connecting member are connected to each other.

Basically, configurations of the present embodiment are substantially the same as those in the above-described first embodiment. A treatment instrument raising wire connection mechanism 20A in the present embodiment is different from the one in the above-described first embodiment in a configuration of a second connecting member 26A and a configuration of a receiving portion 21kA that is provided at a proximal end of a second cylinder 21TA in a cylinder 21A. Other configurations are similar to those in the above-described first embodiment. Therefore, among the configurations of the present embodiment, the same configurations as those in the above-described first embodiment are attached with the same reference signs and descriptions thereof will be omitted, and only different parts will be described in detail below.

As shown in FIG. 13, the treatment instrument raising wire connection mechanism 20A in the endoscope in the present embodiment includes the cylinder 21A, a first moving member 22, a second moving member 25A, and an opening/closing mechanism.

Among these, the cylinder 21A includes a first cylinder 21P and a second cylinder 21TA. The second cylinder 21TA is different from the one in the above-described first embodiment in a proximal end shape of the receiving portion 21kA provided at the proximal end of the second cylinder 21TA. In the present embodiment, the proximal end shape of the receiving portion 21kA is not a tapered shape. In other words, the proximal end shape of the receiving portion 21kA is a cylindrical shape, for example.

The second moving member 25A includes the second connecting member 26A and a treatment instrument raising wire 27. The second connecting member 26A includes a head portion 26Aa and a neck portion 26b. The distal end of the head portion 26Aa is connected to the neck portion 26b.

The head portion 26Aa includes a first inclined portion 26aa and a second inclined portion 26ab. The first inclined portion 26aa is an inclined portion formed in a shape which becomes narrower toward the neck portion 26b. The second inclined portion 26ab is an inclined portion formed in a shape which becomes wider from the proximal end side toward the distal end side (in the direction along the arrow X1) in the head portion 26Aa. Other configurations are the same as those in the above-described first embodiment.

Brief description will be made below on a working of the second embodiment of the present disclosure configured as described above. First, as shown in FIG. 13, in the state where the first moving member 22 and the second moving member 25A are connected to each other, the chuck 24 is arranged such that a part on the proximal end side thereof including a holding portion 24e is located in a small-diameter portion 21b of a first inner space 21m. At this time, the chuck 24 is restricted from being in the open state.

Here, it is supposed that the treatment instrument operation lever 10 has been switched to the setting to operate in the maintenance operation range. If the treatment instrument operation lever 10 is operated in a predetermined direction, the operation force of the treatment instrument operation lever 10 acts on the first moving member 22 of the treatment instrument raising wire connection mechanism 20A through the driving mechanism 30.

This causes the first moving member 22 to move, in the direction along the first axis Ax1, in the direction of the arrow X1 in FIG. 13 by a predetermined distance. At the same time, the chuck 24 also moves in the same direction by the same distance. Then, the holding portion 24e of the chuck 24 passes through the tapered portion 21d to be arranged in the large-diameter portion 21c. The chuck 24 is then brought into a state allowing the release of the head portion 26Aa of the second connecting member 26A.

In this state, when the treatment instrument raising wire 27 is pulled toward the distal end side (in the direction along the arrow X1), for example, the second connecting member 26A moves in the same direction together with the treatment instrument raising wire 27.

At this time, the first inclined portion 26aa of the head portion 26Aa of the second connecting member 26A contacts two claw portions 24b. Then, with the movement of the head portion 26Aa in the direction of the arrow X1, the first inclined portion 26aa opens the chuck 24 to expand the through hole 24f. This allows the head portion 26Aa to be removed from the holding portion 24e. As a result, the connection between the first moving member 22 and the second moving member 25A is released.

On the other hand, the working at the time of connecting the first moving member 22 and the second moving member 25A is as described below.

The second connecting member 26A attached to the proximal end of the treatment instrument raising wire 27 is inserted into the treatment instrument raising wire insertion conduit 33, in the direction along the arrow X2 in FIG. 13. When the proximal end of the second connecting member 26A reaches the treatment instrument raising wire connection mechanism 20A, the second inclined portion 26ab of the head portion 26Aa of the second connecting member 26A comes into contact with the inner surface of the distal-end tapered portion 24a of the chuck 24, to open the chuck 24. This causes the head portion 26Aa to enter the holding portion 24e from the through hole 24f. Since the second inclined portion 26ab is provided, the head portion 26Aa moves smoothly into the holding portion 24e.

In this state, the treatment instrument operation lever 10 is operated in the predetermined direction, to cause the first moving member 22 to move in the direction of the arrow X2 in FIG. 13. Then, the chuck 24 is moved from the large-diameter portion 21c to the small-diameter portion 21b, via the tapered portion 21d. This causes the chuck 24 to change to the closed state. When the chuck 24 is thus arranged in the small-diameter portion 21b, the chuck 24 is restricted from being in the open state.

As described above, the second embodiment is also capable of providing the same effects as those in the first embodiment.

Furthermore, according to the present embodiment, the opening/closing mechanism is configured by further including at least a part of the head portion 26Aa. In the present embodiment, the head portion 26Aa includes the first inclined portion 26aa and the second inclined portion 26ab. The first inclined portion 26aa is formed in the shape which becomes narrower toward the neck portion. The second inclined portion 26ab is formed in the shape which becomes wider from the proximal end side toward the distal end side (in the direction along the arrow X1) in the head portion 26Aa.

The two claw portions 24b of the first connecting member (chuck 24) contacts the first inclined portion 26aa, to open the first connecting member (chuck 24). In addition, the distal-end tapered portion 24a of the first connecting member (chuck 24) contacts the second inclined portion 26ab, to open the first connecting member.

Such a configuration enables smooth opening of the chuck 24 without providing the fourth part (receiving portion 21k) of the cylinder 21, and also enables smooth insertion and removal of the second connecting member 26A into and from the chuck 24.

Next, a third embodiment of the present disclosure will be described below. FIG. 14 is a main-part enlarged cross-sectional view showing, in an enlarged manner, a main part (connecting part between a first connecting member and a second connecting member) of a treatment instrument raising wire connection mechanism in an endoscope in the third embodiment of the present disclosure. Note that FIG. 14 shows a state where the first connecting member and the second connecting member are connected to each other.

Basically, configurations of the present embodiment are substantially the same as those of the above-described first and second embodiments. A treatment instrument raising wire connection mechanism 20B in the present embodiment is different from the one in each of the above-described first and second embodiments in a configuration of a first moving member 22B.

A configuration of a second connecting member 26 is the same as that in the above-described first embodiment. In addition, a configuration of a receiving portion 21kA provided at a proximal end of a second cylinder 21TA in a cylinder 21A is the same as that in the above-described second embodiment. Other configurations are the same as those in the above-described first embodiment. Therefore, among the configurations in the present embodiment, the same configurations as those in the above-described first and second embodiments are attached with the same reference signs and descriptions thereof will be omitted, and only different parts will be described in detail below.

As shown in FIG. 14, the treatment instrument raising wire connection mechanism 20B in the endoscope in the present embodiment includes the cylinder 21A, a first moving member 22B, a second moving member 25, and an opening/closing mechanism.

The first moving member 22B includes a piston rod 23B, a chuck 24, and an elastic member 41. Among these, the elastic member 41 functions as a part of the opening/closing mechanism. The present embodiment shows an example in which the elastic member 41 is included in the first moving member 22B.

The piston rod 23B is the same as the ones in the above-described first and second embodiments in that the chuck 24 which is the first connecting member is disposed at the distal end of the piston rod. Therefore, the piston rod 23B includes, at the distal end thereof, a flange-shaped engaged portion 23b. The engaged portion 23b is engaged with an engaging portion 24d of the chuck 24. Such an engagement between the engaged portion 23b and the engaging portion 24d achieves an integration of the piston rod 23B and the chuck 24.

Further, in the present embodiment, in the engaging portion 24d, the elastic member 41 is attached to the engaged portion 23b.

The elastic member 41 is configured, for example, of a coil spring having a stretchable elastic force. The elastic force of the elastic member 41 acts in a direction for constantly opening a distal-end tapered portion 24a of the chuck 24 as the first moving member 22B.

To this end, the elastic member 41 is arranged in the engaging portion 24d such that it stretches in a direction substantially orthogonal to the first axis Ax1 (direction of the arrow S in FIG. 14). With this, the elastic force of the elastic member 41 acts in the direction substantially orthogonal to the first axis Ax1 (direction of the arrow S in FIG. 14). Accordingly, the elastic force in the direction for constantly opening the chuck 24 is applied to the chuck 24. Other configurations are the same as those in the above-described first embodiment.

Brief description will be made on a working of the third embodiment of the present disclosure configured as described above. First, as shown in FIG. 14, in the state where the first moving member 22B and the second moving member 25 are connected to each other, the chuck 24 is arranged such that a part on the proximal end side thereof including a holding portion 24e is located in a small-diameter portion 21b of a first inner space 21m. At this time, the chuck 24 is restricted from being in the open state. In addition, the elastic member 41 is in a contracted state in the engaging portion 24d.

Here, it is supposed that the treatment instrument operation lever 10 has been switched to the setting to operate in a maintenance operation range. If the treatment instrument operation lever 10 is operated in a predetermined direction, the operation force of the treatment instrument operation lever 10 acts on the first moving member 22B of the treatment instrument raising wire connection mechanism 20B through the driving mechanism 30.

This causes the first moving member 22B to move, in the direction along the first axis Ax1, in the direction of the arrow X1 in FIG. 14 by a predetermined distance. At the same time, the chuck 24 also moves in the same direction by the same distance. Then, the engaging portion 24d of the chuck 24 passes through a tapered portion 21d to be arranged in a large-diameter portion 21c.

When the engaging portion 24d of the chuck 24 is arranged in the large-diameter portion 21c, the chuck 24 receives the elastic force of the elastic member 41, to open, and to be in the state allowing the release of a head portion 26a of the second connecting member 26.

When the treatment instrument raising wire 27 is pulled toward the distal end side (in the direction along the arrow X1), for example, the head portion 26a of the second connecting member 26 is removed from the holding portion 24e, and thereby the connection between the first moving member 22B and the second moving member 25 is released.

When the treatment instrument raising wire 27 is thus pulled in the direction along the arrow X1, the second connecting member 26 moves in the same direction together with the treatment instrument raising wire 27. This allows the head portion 26a to be removed from the holding portion 24e.

On the other hand, the working at the time of connecting the first moving member 22B and the second moving member 25 is as described below.

The second connecting member 26 attached to the proximal end of the treatment instrument raising wire 27 is inserted into the treatment instrument raising wire insertion conduit 33, in the direction along the arrow X2 in FIG. 14. When the proximal end of the second connecting member 26 reaches the treatment instrument raising wire connection mechanism 20B, the head portion 26a of the second connecting member 26 enters the holding portion 24e of the chuck 24 which is in the open state.

In this state, the treatment instrument operation lever 10 is operated in the predetermined direction, to cause the first moving member 22B to move in the direction of the arrow X2 in FIG. 14. This causes the chuck 24 to move from the large-diameter portion 21c to the small-diameter portion 21b, via the tapered portion 21d. With this, the chuck 24 is changed to the closed state against the elastic force of the elastic member 41. When the chuck 24 is thus arranged in the small-diameter portion 21b, the chuck 24 is restricted from being in the open state.

As described above, the third embodiment is capable of providing the same effects as those in the above-described first and second embodiments.

Furthermore, according to the present embodiment, the opening/closing mechanism includes the elastic member 41. In this case, the elastic member 41 is included in the first moving member 22B. The elastic member 41 has the elastic force acting in the direction for opening the first connecting member 24. The elastic member 41 is arranged on the proximal end side of two claw portions 24b.

With such a configuration in which the elastic member 41 is included in the opening/closing mechanism, the chuck 24, along with the movement thereof from the small-diameter portion 21b to the large-diameter portion 21c, receives the elastic force of the elastic member 41, to be changed automatically to the open state. Accordingly, the connected state between the chuck 24 and the second connecting member 26 can be released more easily. In addition, in the state where the first connecting member 24 and the second connecting member 26 are connected to each other, the connected state can be surely maintained.

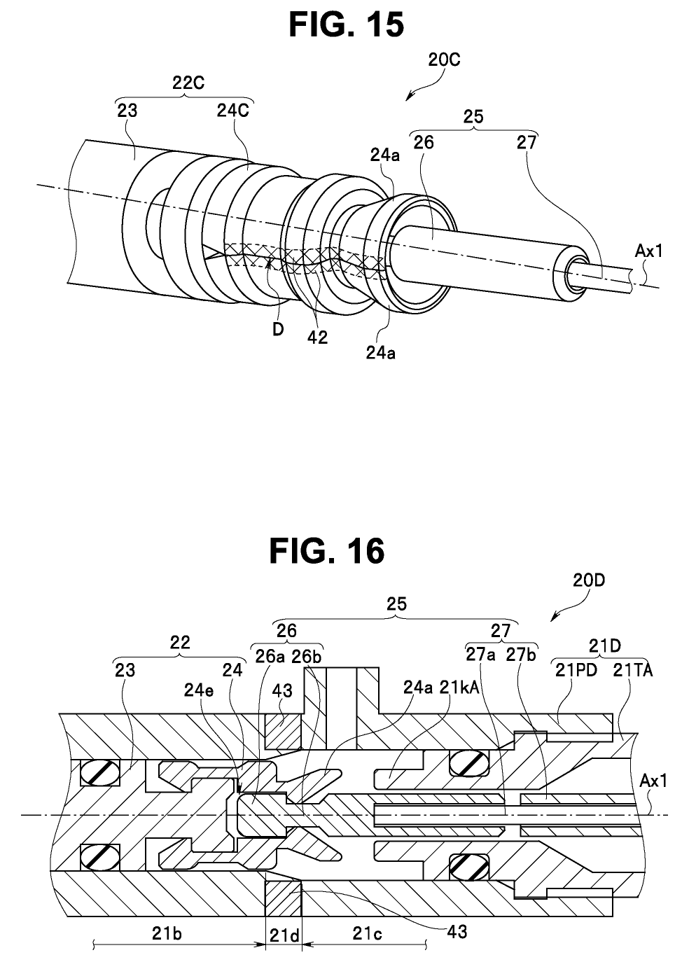

Next, a fourth embodiment of the present disclosure will be described below. FIG. 15 is a main-part enlarged perspective view showing a first connecting member and a second connecting member in a treatment instrument raising wire connection mechanism in an endoscope in the fourth embodiment of the present disclosure. Note that FIG. 15 shows a state where the first connecting member and the second connecting member are connected to each other.

Basically, configurations of the present embodiment are substantially the same as those of the above-described respective embodiments. A treatment instrument raising wire connection mechanism 20C in the present embodiment is different from the ones in the above-described respective embodiments in a configuration of the first connecting member (chuck 24C) in a first moving member 22C. Other configurations are the same as those in the above-described respective embodiments. Therefore, among the configurations in the present embodiment, the same configurations as those in the above-described respective embodiments are attached with the same reference signs and descriptions thereof will be omitted, and only different parts will be described in detail below.

As shown in FIG. 15, in the treatment instrument raising wire connection mechanism 20C in the endoscope in the present embodiment, the first moving member 22C includes a piston rod 23 and the chuck 24C.

The chuck 24C is formed by including magnets 42. The magnets 42 each generate a magnetic force that causes the chuck 24C to open constantly. To this end, each of the magnets 42 is arranged at a predetermined part of each of two members that are formed as two segments of the chuck 24C.

In other words, when the two members, which are formed as the two segments, of the chuck 24C are in the closed state as shown in FIG. 15, the magnets 42 are provided between the proximal end and the distal end along a seam part at which the two members face each other (see the reference sign D in FIG. 15).

In this case, the magnets 42, which are provided respectively to the two members of the chuck 24C, are of the same polarity. Therefore, since a repulsive force of the magnets 42 acts between the two members of the chuck 24C, a force acts between the two members so that they constantly separate from each other. Other configurations are the same as those in the above-described first embodiment.

Note that the magnets 42 are members that function as a part of the opening/closing mechanism. The present embodiment shows an example in which the magnets 42 are included in the first moving member 22C.

The working of the fourth embodiment of the present disclosure configured as described above is substantially the same as that of the third embodiment.

As described above, the fourth embodiment is also capable of providing the same effects as those in the above-described respective embodiments.

Furthermore, according to the present embodiment, the opening/closing mechanism includes the magnets 42. The magnets 42 are provided to the chuck 24C which is the first connecting member of the first moving member 22C. The magnets 42 generate the magnetic force that causes the chuck 24C to open constantly by the repulsive force.

In addition, the magnets 42 include at least two magnets arranged at the respective positions facing each other of the two members of the chuck 24C. The repulsive force of the magnets 42 acts in the direction for opening the chuck 24C constantly.

With such a configuration, when the first moving member 22C is moved from a small-diameter portion 21b to a large-diameter portion 21c, the chuck 24C is changed automatically to the open state due to the magnetic force of the magnets 42. Thus, the connected state between the chuck 24C and the second connecting member 26 can be released easily. In addition, in the state where the chuck 24C and the second connecting member 26 are connected, the connected state can be surely maintained.

Next, a fifth embodiment of the present disclosure will be described below. FIG. 16 is a main-part enlarged cross-sectional view showing a main part (connecting part between a first connecting member and a second connecting member) of a treatment instrument raising wire connection mechanism in an endoscope in the fifth embodiment of the present disclosure. Note that FIG. 16 shows a state where the first connecting member and the second connecting member are connected to each other.

Basically, configurations of the present embodiment are substantially the same as those of the above-described respective embodiments. A treatment instrument raising wire connection mechanism 20D in the present embodiment is different from the ones in the above-described respective embodiments in a configuration of a cylinder 21D. Other configurations are the same as those in the above-described respective embodiments. Therefore, among the configurations in the present embodiment, the same configurations as those in the above-described respective embodiments are attached with the same reference signs and descriptions thereof will be omitted, and only different parts will be described in detail below.

As shown in FIG. 16, in the treatment instrument raising wire connection mechanism 20D in the endoscope in the present embodiment, the cylinder 21D includes a first cylinder 21PD and a second cylinder 21TA.

Of these, the first cylinder 21PD includes magnets 43. The magnets 43 are provided to at least one of a second part (large-diameter portion 21c) or a third part (tapered portion 21d). In this case, the magnets 43 are provided respectively at positions facing each other across a first inner space 21m (21a) in the first cylinder 21PD. Furthermore, the respective magnets 43 are provided respectively to the two members of the chuck 24 at the positions facing each other.

As described above, the chuck 24 is formed of a material such as metal. Therefore, a force acting in a direction for constantly opening the chuck 24 due to the magnetic force of the magnets 43 is applied to the chuck 24.

Meanwhile, similar to the above-described second, third, and fourth embodiments, a second cylinder 21TA is configured such that a proximal end shape of a receiving portion 21kA has a cylindrical shape, for example, and does not have a tapered shape. Other configurations are the same as those in the above-described first embodiment.

Note that the magnets 43 are members that function as a part of an opening/closing mechanism. The present embodiment shows an example in which the magnets 43 are included in the first cylinder 21PD of the cylinder 21D.

The working of the fifth embodiment of the present disclosure configured as described above is substantially the same as those of the above-described third and fourth embodiments.

As described above, the fifth embodiment is also capable of providing the same effects as those in the above-described respective embodiments.

Furthermore, according to the present embodiment, the opening/closing mechanism includes the magnets 43. The magnets 43 are provided to at least one of the second part (large-diameter portion 21c) or the third part (tapered portion 21d). An attraction force of the magnets 43 causes the chuck 24 to open.

With such a configuration, when the first moving member 22C is moved from the small-diameter portion 21b to the large-diameter portion 21c, the chuck 24 is changed automatically to the open state due to the magnetic force of the magnets 43. Therefore, the connected state between the chuck 24 and the second connecting member 26 can be released easily. In addition, in the state where the chuck 24 and the second connecting member 26 are connected to each other, the connected state can be surely maintained.

Note that the above-described respective embodiments each show the example in which the chuck 24, which is the first connecting member, is configured of the two members formed as the two segments. However, the configuration of the chuck is not limited to that in the example. For example, the chuck may be configured of multiple members formed as multiple segments.

Incidentally, in the endoscope in each of the above-described respective embodiments, the treatment instrument raising wire connection mechanism is provided between the treatment instrument operation lever and the treatment instrument raising base, to thereby achieve the mechanism which enables easy attachment and detachment of the treatment instrument raising wire.

This achieves the configuration that enables, for example, the treatment instrument raising base and the treatment instrument raising wire to be replaced in the endoscope easily by a simple operation. Such a configuration facilitates the achievement of the configuration in which the treatment instrument raising base and the treatment instrument raising wire are replaced every time the endoscope is used. The endoscope shown in each of the respective embodiments is supposed to be the endoscope having such a configuration.

However, the present disclosure is not limited to the configurations shown in each of the above-described embodiments. For example, the present disclosure can be similarly applied even to an endoscope configured such that only the treatment instrument raising wire is replaced for each use of the endoscope.

In addition, the present disclosure can be similarly applied even to an endoscope configured such that the distal end portion including the treatment instrument raising base, and the treatment instrument raising wire are replaced for each use of the endoscope. Furthermore, the present disclosure can be similarly applied even to an endoscope configured such that the entire insertion portion is replaced for each use of the endoscope.

The present disclosure is not limited to the above-described embodiments, and it goes without saying that various modifications and applications can be implemented within a range without departing from the subject matter of the disclosure. Furthermore, the above embodiments include disclosures at various stages, and various disclosures can be extracted by appropriate combinations of a plurality of disclosed constituent elements. For example, even when some of the constituent elements are removed from all the constituent elements shown in the above-described one embodiment, a configuration from which the constituent elements are eliminated can be extracted as a disclosure insofar as the configuration can solve the problem to be solved by the disclosure and attain the effects of the disclosure. Furthermore, components over different embodiments may be combined as appropriate. The disclosure is not limited by the specific embodiments except as limited by appended claims.

The present application claims the benefit of U.S. Patent Application No. 63/536,782 filed in the United States on September 6, 2023. The contents disclosed in the above-described basic application are incorporated in the description, claims, and drawings in the present application by reference.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:

Clause 1: An endoscope comprising:

a cylinder;

a first moving member;

a second moving member; and

an opening/closing mechanism, wherein:

the cylinder includes an inner space;

the inner space is formed along a first axis extending from a distal end toward a proximal end;

the first moving member is configured to be movable in the inner space along the first axis and includes a first connecting member;

the first connecting member includes at least two protrusions configured to catch and hold the second moving member;

the second moving member is configured to be movable in the inner space along the first axis and includes a second connecting member;

the second connecting member is held by the first connecting member;

the second connecting member includes a head portion configured to be held by the first connecting member and a neck portion provided on a distal end side of the head portion and formed to be narrower than the head portion;

the opening/closing mechanism changes a state of the first connecting member between a state where the first connecting member is opened to release the second connecting member and a state where the first connecting member is closed to hold the second connecting member;

the opening/closing mechanism is at least a part of the cylinder;

the inner space includes a first part and a second part;

the first part has a first cross-sectional area;

the first cross-sectional area has a size that does not allow for the first connecting member to release the second connecting member;

the second part is provided on a distal end side of the first part and has a second cross-sectional area larger than the first cross-sectional area; and

the second cross-sectional area has a size that allows for the first connecting member to release the second connecting member.

Clause 2: The endoscope according to clause 1, wherein

the inner space further includes a third part,

the third part is a tapered portion that connects the first part and the second part, and

the tapered portion contacts the first connecting member to close the first connecting member.

Clause 3: The endoscope according to clause 1, wherein

the cylinder further includes a fourth part,

the fourth part includes a receiving portion provided in the second part and formed in a shape expanding toward the distal end side, and

the receiving portion contacts the first connecting member to open the first connecting member.

Clause 4: The endoscope according to clause 1, wherein

the opening/closing mechanism further includes at least a part of the head portion,

the head portion includes a first inclined portion formed in a shape which becomes narrower toward the neck portion, and

the first connecting member contacts the first inclined portion, and the first connecting member is opened.

Clause 5: The endoscope according to clause 4, wherein

the head portion further includes a second inclined portion,

the second inclined portion is formed in a shape which becomes wider from a proximal end side toward the distal end side in the head portion, and

the first connecting member contacts the second inclined portion, and the first connecting member is opened.

Clause 6: The endoscope according to clause 1, wherein

the opening/closing mechanism further includes an elastic member, and

the elastic member is provided in the first moving member and has an elastic force in a direction for opening the first connecting member.

Clause 7: The endoscope according to clause 6, wherein

the elastic member is arranged on a proximal end side of the two protrusions.

Clause 8: The endoscope according to clause 1, wherein

the opening/closing mechanism further includes magnets, and

the magnets are provided at the first moving member and generate a magnetic force to open the first connecting member.

Clause 9: The endoscope according to clause 8, wherein

the magnets include at least two magnets arranged at the first connecting member such that like poles of the magnets face each other, and the first connecting member is opened by a repulsive force of the magnets.

Clause 10: The endoscope according to clause 2, wherein

the opening/closing mechanism further includes magnets, and

the magnets are provided to at least one of the second part or the third part, and the first connecting member is opened by an attractive force of the magnets.

Clause 11: The endoscope according to clause 1, wherein

the endoscope comprises an operation portion and an insertion portion,

the insertion portion is provided on a distal end side of the operation portion, and

the operation portion includes the cylinder, the first moving member, the second moving member, and the opening/closing mechanism.

Clause 12: The endoscope according to clause 11, wherein

the insertion portion includes a bending portion,

the operation portion includes an angle knob,

the bending portion actively bends by the angle knob being operated, and

the first connecting member is positioned on the distal end side with respect to the angle knob.

Clause 13: The endoscope according to clause 11, wherein

the first moving member includes a lever on a proximal end side of the first connecting member, and

the lever is rotationally moved, to thereby cause the first connecting member to move in the inner space.

Clause 14: The endoscope according to clause 13, wherein

the second moving member includes a wire and a raising base,

the wire is provided on a distal end side of the neck portion,

the raising base is provided on a distal end side of the wire, and

the raising base rotationally moves with the rotational movement of the lever.

Clause 15: The endoscope according to clause 1, wherein

the first moving member includes a lever on a proximal end side of the first connecting member,

the lever is rotationally moved, to thereby cause the first connecting member to move in the inner space,

the lever includes a switching mechanism configured to switch a range within which the lever rotationally moves, between a normal operation range and a maintenance operation range exceeding the normal operation range,

when the lever is in the normal operation range, the first connecting member is positioned in the first part, and

when the lever is in the maintenance operation range, the first connecting member is positioned in the second part.

What is claimed is:

1. An endoscope comprising:
a cylinder including an inner space formed along an axis extending from a distal end toward a proximal end;
a first moving member configured to move in the inner space along the axis and including a first connecting member configured to be openable and closable, the first connecting member including at least two protrusions;
a second moving member configured to move in the inner space along the axis and including a second connecting member configured to be held by the first connecting member, the second connecting member including:
a head portion configured to be held by the at least two protrusions; and
a neck portion disposed closer to the distal end than the head portion is and formed to be narrower than the head portion; and
an opening/closing mechanism configured to change a state of the first connecting member between an open state where the first connecting member is opened to release the second connecting member and a closed state where the first connecting member is closed to hold the second connecting member, the opening/closing mechanism including:
a first part having a first cross-sectional area adapted in size to restrict the first connecting member from releasing the second connecting member; and
a second part disposed closer to the distal end than the first part is, and having a second cross-sectional area that is larger than the first cross-sectional area and adapted in size to allow the first connecting member to release the second connecting member, wherein the first part and the second part form at least a part of the inner space, and the opening/closing mechanism forms at least a part of the cylinder.

2. The endoscope according to claim 1, wherein
the opening/closing mechanism further includes a third part that is a tapered portion disposed between the first part and the second part and configured to contact the first connecting member to close the first connecting member, the first, second, and third parts forming at least a part of the inner space.

3. The endoscope according to claim 1, wherein
the opening/closing mechanism further includes a receiving portion disposed in the second part, having a shape expanding toward the distal end, and configured to contact the first connecting member to open the first connecting member.

4. The endoscope according to claim 1, wherein
the opening/closing mechanism further includes at least a part of the head portion,
the head portion includes a first inclined portion having a shape tapering toward the neck portion and configured to contact the first connecting member to open the first connecting member.

5. The endoscope according to claim 4, wherein
the head portion further includes a second inclined portion having a shape expanding toward the distal end and configured to contact the first connecting member to open the first connecting member.

6. The endoscope according to claim 1, wherein
the opening/closing mechanism further includes an elastic member disposed in the first moving member and configured to exert an elastic force in a direction for opening the first connecting member.

7. The endoscope according to claim 6, wherein
the elastic member is disposed closer to the proximal end than the two protrusions are.

8. The endoscope according to claim 1, wherein
the opening/closing mechanism further includes at least one magnet disposed at the first moving member and configured to generate a magnetic force to open the first connecting member.

9. The endoscope according to claim 8, wherein
the at least one magnet includes at least two magnets disposed, at the first connecting member, to have like poles facing each other, thereby opening the first connecting member by a repulsive force of the at least two magnets.

10. The endoscope according to claim 2, wherein
the opening/closing mechanism further includes at least one magnet provided to at least one of the second part or the third part, thereby opening the first connecting member by an attractive force of the at least one magnet.

11. The endoscope according to claim 1, further comprising:
an operation portion including the cylinder, the first moving member, the second moving member, and the opening/closing mechanism; and
an insertion portion disposed at a distal end part of the operation portion.

12. The endoscope according to claim 11, wherein
the operation portion includes an angle knob,
the insertion portion includes a bending portion configured to actively bend in response to the angle knob being operated, and
the first connecting member is positioned closer to the distal end than the angle knob is.

13. The endoscope according to claim 11, wherein
the first moving member further includes a lever disposed closer to the proximal end than the first connecting member, and
the first connecting member is configured to move in the inner space in response to the lever being rotated.

14. The endoscope according to claim 13, wherein
the second moving member further includes:
a wire disposed closer to the distal end than the neck portion is;
a raising base disposed closer to the distal end than the wire is and configured to rotate in response to the lever being rotated.

15. The endoscope according to claim 1, wherein
the first moving member further includes a lever disposed closer to the proximal end than the first connecting member is,
the first connecting member is configured to move in the inner space in response to the lever being rotated,
the lever includes a switching mechanism configured to switch a movable range of the lever between a normal operation range and a maintenance operation range exceeding the normal operation range,
when the lever is in the normal operation range, the first connecting member is positioned in the first part, and
when the lever is in the maintenance operation range, the first connecting member is positioned in the second part.

16. The endoscope according to claim 1, wherein
the first connecting member includes two members formed by dividing a cylindrical member along a plane including the first axis, and
the two members are configured to move in a direction to separate from each other, thereby opening the first connecting member.

17. The endoscope according to claim 1, wherein
the cylinder includes a first cylinder and a second cylinder configured to be integrally connected to each other, and
a sealing member is disposed in at least one of a gap between an inner circumferential surface of the first cylinder and an outer circumferential surface of the first moving member, or a gap between the inner circumferential surface of the first cylinder and an outer circumferential surface of the second cylinder.

18. The endoscope according to claim 14, wherein
the wire includes a core wire and a covering tube covering the core wire, and
the covering tube is configured to fill a gap between an outer surface of the core wire and an inner surface of a conduit through which the wire is inserted.

19. The endoscope according to claim 17, wherein the first cylinder and the second cylinder are threadedly engaged with each other to be integrally connected.

20. The endoscope according to claim 11, wherein
the cylinder includes an adjusting screw portion formed thereon,
the operation portion includes a fixing portion including a screw portion formed thereon, and
an axial position of the cylinder relative to the fixing portion is adjustable by adjusting a state of threaded engagement between the adjusting screw portion and the screw portion that are threadedly engaged with each other.
